(19) United States
(12) Patent Application Publication
(10) Pub. No.: US 2008/0031832 A1
(43) Pub. Date: Feb. 7, 2008

(54) Surface-Doped Particles Of Ti02 Or Zno And Their Use

(75) Inventors: Gareth Wakefield, Oxford (GB); George Barry Park, Oxford (GB); Sarah Lipscomb, Oxford (GB); Edward Holland, Oxford (GB)

Correspondence Address: WOLF GREENFIELD & SACKS, P.C., 600 ATLANTIC AVENUE, BOSTON, MA, 02210-2206, US

(73) Assignee: Oxonica Limited, Kidlington,, Oxford (GB)

(21) Appl. No.: 10/588,071

(22) PCT Filed: Jan. 26, 2005
(86) PCT No.: PCT/GB05/00257

§ 371 Date: May 7, 2007

(30) Foreign Application Priority Data
Jan. 28, 2004 (GB) 0401838.8
Jan. 28, 2004 (GB) 0401840.4
Jan. 28, 2004 (GB) 0401841.2
Jan. 28, 2004 (GB) 0401846.1
Mar. 5, 2004 (GB) 0405078.7

(51) Int. Cl. C09C 1/36; A01N 25/22; A61K 8/42; C01G 23/00; C01G 9/00; C09C 1/04

(52) U.S. Cl. 424/59; 106/287.19; 423/610; 423/622; 424/641; 427/372.2; 524/413; 524/432

(57) ABSTRACT

A particle of TiO₂ or ZnO which has been doped with one or more other elements such that the concentration of dopant in the surface of the particle is greater than that at the core of the particle, and compositions containing such particles for use as sunscreens or in veterinary, agricultural or horticultural compositions or as coatings for plastics and other materials.

The present invention relates to novel particles which find utility as degradation protectors, for example in UV screening compositions suitable for cosmetic and topical pharmaceutical use, for use in agriculture, horticulture and veterinary medicine, and for mechanical, structural or environmental protection in the form for example of plastic articles, paints and varnishes.

In our British Application No. 0315082.8, structural or we describe how the degradation of organic sunscreen agents, and other components which are susceptible to degradation, can be retarded if the compositions also have present zinc oxide or titanium dioxide which has been doped with another element and/or reduced zinc oxide. These can be regarded as degradation protectors because they help to protect sunscreen ingredients which are unstable to sunlight against sunlight-induced photo-degradation. By using these doped or reduced materials rather than ordinary titanium dioxide or zinc oxide it is, for example, possible either to provide a composition which gives better protection against UV light for the same quantity of organic sunscreen agent or a composition having the same screening effect against UV light but containing a smaller quantity of organic sunscreen agent. Indeed it is possible to provide all day protection sunscreens by incorporating the doped and/or reduced materials. Sometimes the degradation products (breakdown chemicals) are toxic.

The invention will be disclosed in terms of four embodiments, although it is expressly stated here that any features from two or more of these embodiments may be combined. The first embodiment concerns particles themselves; the second embodiment concerns compositions for cosmetic and topical pharmaceutical use, for example for use as UV sunscreens, the third embodiment concerns compositions for use in providing mechanical, structural or environmental protection; and the fourth embodiment concerns compositions suitable for veterinary, agricultural or horticultural use.

### The First Embodiment

The first embodiment of the invention provides a particle of TiO₂ or ZnO which has been doped with one or more other elements such that the concentration of dopant in a surface of the particle is greater than that at a core of the particle.

The expression “in the surface”, as used herein, means, assuming a substantially spherical particle, the outer shell which has a thickness not exceeding 10% of the radius of the particle. It will be appreciated that the presence of dopant “in the surface” which includes “at the surface” is to be contrasted with material which can be on the surface as in the case of a simple coating. “At the surface” means dopant which is bound to the particle other than by pure electrostatic forces as is the case with a coating. As used herein, the term “the core” means, assuming a substantial spherical particle, the sphere at the centre of the particle whose radius does not exceed 10% of the radius of the particle (or, in the case of substantially non-spherical particles, 10% of the largest dimension). The term “bulk of the particle” means the particle excluding the said outer shell.

It is preferred that the concentration of dopant in the surface of the particle is greater than that in the bulk of the particle and it is particularly preferred that there is no dopant at the core of the particle. In other words, there will be a concentration gradient e.g. such that the ratio of dopant atoms to titanium or zinc atoms in the surface is greater than the ratio in the core or centre where it may be zero.

The optimum total amount of the second component on the particle may be determined by routine experimentation, but it is preferably low enough so that the particles are minimally coloured. Amounts as low as 0.1 mole % or less, for example 0.05 mole %, or as high as 1 mole % or above, for example 5 mole % or 10 mole %, can generally be used. Typical concentrations are from 0.5 to 2 mole % by weight. The mole ratio of dopant to host metal on the surface is typically from 2-25:98-75, usually 5-20:95-80 and especially 8-15:92-85. The amount of dopant at the surface can be determined by, for example, X-ray Photoelectron Spectroscopy (XPS).

Suitable dopants for the oxide particles include manganese, which is especially preferred, e.g. Mn²⁺ but especially Mn³⁺, vanadium, for example V³⁺ or V⁵⁺, chromium, cerium, selenium and iron but other metals which can be used include nickel, copper, tin, e.g. Sn⁴⁺, aluminium, lead, silver, zirconium, zinc, cobalt, e.g. Co²⁷, gallium, niobium, for example Nb⁵⁺, antimony, for example Sb³⁺, tantalum, for example Ta⁵⁺, strontium, calcium, magnesium, barium, molybdenum, for example Mo³⁺, Mo⁵⁺ or Mo⁶⁺ as well as silicon. These metals can be incorporated singly or in combinations of two or three or more. It will be appreciated that for effective bulk doping the size of the ion must be such as can readily be inserted into the crystal lattice of the particle. On the other hand there is no such size limitation for the elements used in surface doping; preferred surface dopants include manganese, eg. as Mn²⁺, cerium, selenium, iron, chromium and vanadium.

The surface-doped particles of the present invention can be obtained by any one of the standard processes for preparing such doped oxides and salts. Titanium oxide and zinc oxide are generally doped by two basic methods involving either coprecipitation or absorption, although other processes including flame pyrolysis can be used provided there is sufficient dopant at the surface. It will be appreciated that coprecipitation will generally result in a fairly uniform distribution of dopant throughout the particle with a result that such procedures are generally not suitable for preparing the particles of the present invention. On the other hand, absorption processes can readily be used provided that the process is stopped before the dopant becomes absorbed substantially uniformly to the core. In other words, if the procedure is stopped at a stage earlier than one would normally use to obtain doped material then one can obtain particles where the concentration of dopant is greater in the surface than at the core.

This can be achieved by using, for example, shorter reaction times. It will be appreciated that the dopant need not necessarily be present as an oxide but may be present as a salt such as a chloride or salt with an oxygen-containing anion such as perchlorate or nitrate. Such techniques include a baking technique by combining particles of a host lattice (TiO₂/ZnO) with a second component in the form of a salt such as a chloride or an oxygen-containing anion such as a perchlorate or a nitrate, in solution or suspension, typically in solution in water, and then baking it, typically at a temperature of at least 300° C. and then calcining it at a higher temperature, for example at least 500° or 600° C. Accordingly the present invention provides a process for preparing the particles of the present invention which comprises placing a particle of TiO₂ or ZnO in contact with a solution or suspension of a salt of the dopant for a time insufficient for the concentration of dopant salt in the core of the particle to reach that at its surface and then baking the resulting particle.

It will be appreciated that such baking techniques and the like will result in dopant in the surface forming part of the crystal lattice while in coating the dopant will remain as a separate layer on the particle surface. It may well be the case that if the dopant is to quench internally generated free radicals effectively then it needs to be in the crystal lattice.

The rutile form of titania is known to be less photoactive than the anatase form and is therefore preferred.

The zinc oxide subjected to surface doping can be reduced zinc oxide. Reduced zinc oxide particles (i.e. particles which possess an excess of zinc ions relative to the oxygen ions) may be readily obtained by heating zinc oxide particles in a reducing atmosphere to obtain reduced zinc oxide particles which absorb TV light, especially UV light having a wavelength below 390 nm, and re-emit in the green, preferably at about 500 nm. Typically the concentration of hydrogen is from 1 to 20%, especially 5 to 15%, by volume, with the balance inert gas, especially nitrogen. A preferred reducing atmosphere is about 10% hydrogen and about 90% nitrogen by volume. The zinc oxide is heated in this atmosphere at, say, 500° to 1000° C., generally 750 to 850° C., for example about 800° C., for 5 to 60 minutes, generally 10 to 30 minutes. Typically it is heated to about 800° C. for about 20 minutes. It will be understood that the reduced zinc oxide particles will contain reduced zinc oxide consistent with minimising migration to the surface of the particles of electrons and/or positively charged holes such that when said particles are exposed to UV light in an aqueous environment the production of hydroxyl radicals is substantially reduced as discussed above.

It is believed that the reduced zinc oxide particles possess an excess of Zn²⁺ ions within the absorbing core. These are localised states and as such may exist within the band gap. A further discussion of this can be found in WO 99/60994.

The average primary particle size of the particles is generally from about 1 to 200 nm, for example about 1 to 150 nm, preferably from about 1 to 100 nm, more preferably from about 1 to 50 nm and most preferably from about 20 to 50 nm. The particle size is preferably chosen to prevent the final product from appearing coloured. Thus nanoparticles are frequently used. Since the scavenging effect is believed to be essentially catalytic it is desirable that the particles are as small as possible to maximise their surface area and hence the area of doped material on the surface. This small size has the advantage that less dopant is needed which has the consequential advantage that any colouring effect caused by the dopant is reduced. However, in one embodiment slightly larger particles for example from 100 to 500 nm, typically 100 to 400 or 450 mm especially from 150 to 300 nm and particularly 200 to 250 nm, can be employed. These provide good coverage of, for example, skin imperfections without unacceptable skin whitening.

Where particles are substantially spherical then particle size will be taken to represent the diameter. However, the invention also encompasses particles which are non-spherical and in such cases the particle size refers to the largest dimension.

The oxide particles of the present invention may have an inorganic or organic coating. For example, the particles may be coated with oxides of elements such as aluminium, zirconium or silicon, especially silica or, for example, aluminium silicate. The particles of metal oxide may also be coated with one or more organic materials such as polyols, amines, alkanolamines, polymeric organic silicon compounds, for example, RSi[{OSi(Me)₂}xOR¹]₃ where R is C₁-C₁₀ alkyl, R¹ is methyl or ethyl and x is an integer of from 4 to 12, hydrophilic polymers such as polyacrylamide, polyacrylic acid, carboxymethyl cellulose and xanthan gum or surfactants such as, for example, TOPO. If desired the surface doping can be carried out by a coating technique either separately or in combination with the inorganic or organic coating agent. Thus for example the undoped oxide can be coated with, say, manganese oxide along with an organic or inorganic coating agent such as silica. It is generally unnecessary to coat the oxide particles to render them hydrophilic, so that for the aqueous phase the particles can be uncoated. However if the particles are to be in the organic or oily phase their surface needs to be rendered hydrophobic or oil-dispersible. This can be achieved by the application directly of, for example, a suitable hydrophobic polymer or indirectly by the application of a coating, for example of an oxide such as silica (which imparts a hydrophilic property) to which a hydrophobic molecule such as a metal soap or long chain (e.g. C₁₂-C₂₂) carboxylic acid or a metal salt thereof such as stearic acid, a stearate, specifically aluminium stearate, aluminium laurate and zinc stearate.

It should be understood that the term “coating” is not to be construed as being limited to a complete covering. Indeed it is generally beneficial for the coating not to be complete since the coating can act as a barrier to the interaction of the free radicals with the dopant on or in the surface of the particle. Thus it is preferred that the coating should be discontinuous where maximum scavenging effect is desired. However it will be appreciated that dopant on the surface can still act to quench free radicals generated within the particle in which case the coating can be continuous. Since coatings of silanes and silicones which can be polymeric or short chain or monomeric silanes are generally continuous these are generally less preferred. Thus coating with an inorganic oxide is generally preferred since these generally do not result in a complete coating on the surface of the particles.

Typical coating procedures include the deposition of silica by mixing alkali such as ammonium hydroxide with an orthosilicate, such as tetraethylorthosilicate, in the presence of the particle. Alternatively the particle can first be coated with a silane such as (3-mercaptopropyl)trimethoxy silane (MPS) and then silicate e.g. sodium silicate is added. The silane attaches to the particle surface and acts as a substrate for the silicate which then polymerises to form silica. Similar techniques can be used for other inorganic oxides.

The particles of the present invention can be used in all the compositions described in our co-pending British Patent Application referred to above, and they are also useful in the polymer and agricultural compositions described in our further co-pending British Patent Applications also filed on the same day as this application and entitled Improved Polymeric Composition and Improved Agricultural Compositions.

The following Example further illustrates the present invention.

### EXAMPLE 1

### Acid Extraction of Manganese Doped Titania

Samples of manganese doped titania were soaked in 25% hydrochloric acid for various times at room temperature. The titania was settled by centrifugation and the supernatant liquid transferred to a 50 ml volumetric flask. The titania was washed once by re-suspension in water with the aid of ultrasonics and again centrifuged. The washings were added to the volumetric flask and the contents made to 50 ml. with de-ionised water.

Samples of the extracts, together with the original powder samples, were analysed for manganese. The water extracts were analysed directly by AAS (Atomic Absorption Spectroscopy). The powders were similarly analysed, after digestion with a hydrofluoric acid-sulphuric acid mixture.

### DPPH (Radical Scavenging) Assay

A stock solution of 1 mM DPPH in MeOH was made. Samples containing 120 μl of DPPH (1 mM) plus 300 μl TiO₂ (3 mg/ml) were made up to 3 ml with MeOH and were placed in a 10 mm quartz cuvette. DPPH is a stable radical, which absorbs at 520 nm, therefore a loss of absorbance at this wavelength, is a measure of the radical scavenging ability of the TiO₂. The titania samples were taken from the above series of extractions. The samples were kept in the dark and the absorbance at 520 nm measured every 5 minutes. The samples required mixing before each measurement was taken in order to redisperse the TiO₂.

| Time of exposure | Extracted Mn | Rate of loss of DPPH |
|---|---|---|
| (hrs) | (%) | (mAbs/min) |
| 0 | 0 | 3.4 |
| 0.25 | 3.22 | 2.05 |
| 1.5 | 4.58 | 1.6 |
| 48 | 26.0 | 0.35 |

It is clear from these data that 74% of the manganese remained after 48 hours. As the rate of loss of DPPH is then very small it is clear that it is the remaining 26% of the manganese which is in or on the surface which acts to scavenge free radicals. Thus particles having manganese available at the surface will scavenge free radicals.

### The Second Embodiment

The second embodiment of the present invention relates to degradation protectors in UV screening compositions suitable for cosmetic and topical pharmaceutical use.

The effects associated with exposure of the skin to UVA and UVB light are well known and include, for example, sunburn, premature ageing and skin cancer.

Commercial sunscreens generally contain components which are able to reflect and/or absorb UV light. These components include, for example, inorganic oxides such as zinc oxide and titanium dioxide as well as organic sunscreen agents.

The general public are generally more concerned by the obvious effects of sunlight, namely sunburn which causes reddening of the skin than they are with other effects of sunlight which are less self evident. As a consequence of this commercial sunscreen compositions are rated by a Sun Protection Factor (SPF). This is a measure of the time taken for skin to redden under a layer of the composition as compared with untreated skin. Thus an SPF of 20 indicates that skin will take 20 times longer to redden under a layer of the composition applied at 2 mg per cm² compared with untreated skin. This reddening effect is caused principally by UVB light. There is no recognised corresponding factor for the effects of UVA light even though the latter may be more damaging in the long term.

Most organic sunscreen agents absorb light over only a part of the UVA-UVB spectrum with the result that if one is to obtain a screening effect covering the whole UVA-UVB spectrum it is generally necessary to use a combination of different organic sunscreen agents. Some organic sunscreen agents and other components of sunscreen compositions are stable to UV light but others are photosensitive and/or may after being excited by UV light result in the formation of free radicals which may attack and cause degradation of another ingredient of the composition.

Titanium dioxide and zinc oxide are generally formulated as “micronised” or “ultrafine” (20-50 nm) particles (so-called microreflectors) because particles whose size is less than 10% of the wavelength of the incident light scatter light according to Rayleigh's Law, whereby the intensity of scattered light is inversely proportional to the fourth power of the wavelength. Consequently, they scatter UVB light (with a wavelength of from 280 or 290 to 315/320 nm) and UVA light (with a wavelength of from 315/320 to 400 nm) more than the longer, visible wavelengths, preventing sunburn whilst remaining invisible on the skin.

However, titanium dioxide and zinc oxide also absorb UV light efficiently, leading via the initial formation of electron hole pairs to the formation of superoxide and hydroxyl radicals in contact with water, which may in turn initiate damage to other components of the composition. The crystalline forms of TiO₂, anatase and rutile, are semiconductors with band gap energies of about 3.23 and 3.06 eV respectively, corresponding to light of about 385 nm and 400 nm (1 eV corresponds to 8066 cm⁻¹). Indeed there is evidence to suggest that TiO₂ can enhance the degradation of organic sunscreen agents, including UVA organic sunscreens, for example avobenzone (butyl methoxydibenzoyl methane, also known as BMDM). Attempts have been made to reduce the adverse effects of TiO₂ and ZnO by coating but coatings are not invariably effective.

The reason why most sunscreen agents do not have a substantially perpetual effect (i.e. an SPF factor which remains substantially constant) is principally because the organic sunscreen agents are degraded by light and/or are adversely affected by other components of the sunscreen composition once the latter are subjected to UV light.

Accordingly, the present invention also provides method of reducing the production of toxic compounds in a UV sunscreen composition which comprises incorporating therein a doped TiO₂/ZnO and/or reduced ZnO. In general the composition containing the doped TiO₂/ZnO has a rate of loss of UV absorption at least 5% preferably at least 10%, more preferably at least 15%, especially at least 20% and most preferably at least 40%, less than that of a composition having the same formulation except that it does not contain the doped material. Thus if the rate of loss of UV absorption (during UV exposure) over at least a proportion of the UVA and/or UVB spectrum is X then the amount of the organic component(s) which are photosensitive and/or which are degraded by another ingredient of the composition possesses a said rate of loss of Y where Y is greater than X by at least 5%, and the amount of doped TiO₂ and/or ZnO reduces the said rate of loss from Y to X.

It has now been appreciated, according to the present invention, that it is important that if the oxide is to be really effective there must be dopant on its surface which can interact with the component of the composition to be protected. For example if, in a two phase composition, the oxide is present in the aqueous phase and the component to be protected is in the organic phase there is little interaction because of the phase boundary; the oxide is not accessible to the component. Thus the free radicals generated by degradation of the component cannot contact the dopant without moving from one phase to another. It has further been realised that if the dopant is solely in the bulk it is not able to interact effectively (as a free radical scavenger) with the component of the composition to be protected. A consequence of this is that it is possible to use materials which are only surface doped i.e where there is dopant in or on the surface of the particle. In one embodiment such materials may be used in a single phase formulation. Although the presence of bulk dopant is desirable where the composition is intended to protect the skin, because the dopant is able to trap free radicals generated by the action of UV light and dissipate the energy produced, this is not essential for a formulation which is not intended to have a skin protection effect. It should be added that the effect of bulk dopant occurs regardless of the phase in which the particle is placed, in contrast to surface dopant.

Accordingly the present invention provides (although not dependant on the above theory) a TV sunscreen composition suitable for cosmetic or topical pharmaceutical use which comprises: (a) one or more organic components which are photosensitive and/or which are susceptible to degradation by another ingredient of the composition and/or by undoped TiO₂ and/or by undoped ZnO; and (b) TiO₂ and/or ZnO which has been surface doped with one or more other elements, typically one i.e. a second element. Where the particle has been bulk doped there will, in general, be dopant throughout the particle. On the other hand where the particle has been “surface doped” (i.e. the dopant is only in or on the surface) there will be a concentration gradient e.g. such that the ratio of dopant atoms to titanium or zinc atoms at the surface or outmost “skin” of the particle is greater than the ratio in the core or centre where it may be zero.

By “UV sunscreen composition suitable for cosmetic or topical pharmaceutical use” is meant any cosmetic or topical pharmaceutical composition having UV sunscreen activity i.e. it includes compositions whose principal function may not be sunscreening. It will be appreciated that the doped TiO₂/ZnO or reduced ZnO may be the only ingredient of the composition having UV sunscreen activity i.e. the composition need not necessarily contain an organic UV sunscreen agent. It is to be understood that the composition can also contain TiO₂ and/or ZnO which has not been doped or reduced.

The organic component which is photosensitive or degraded by another ingredient of the composition is generally a UV sunscreen agent. Although all organic sunscreen agents which suffer a loss in UV absorption can be used, the present invention is particularly useful for agents which absorb in the UVA region as well as in the UVB region. However, other organic components will generally be susceptible to free radical attack and in turn this generally may cause degradation of the UV sunscreen agent.

As indicated above the UV absorption of an organic sunscreen agent generally decreases with time. In contrast, the UV absorption of TiO₂ or ZnO does not decrease with time. Since TiO₂ and ZnO absorb in both the UVA and UVB region whereas an organic sunscreen agent is generally more wavelength specific, it can be seen that the UVA/UVB absorption ratio may change over time. For example, as is preferred, where the organic sunscreen agent absorbs in the UVA region, then the ratio will decrease over time. When doped TiO₂/ZnO is used, rather than the same quantity of undoped TiO₂/ZnO, the rate of change is reduced. This is because the doped material will enhance the performance of the organic sunscreen agent over time relative to the situation where undoped TiO₂/ZnO is present. Thus with a UVA sunscreen the loss of UVA absorption over time is reduced (i.e. the UVA response is more stable when the doped material is present) so that the ratio of change of the rates is reduced. Thus if the initial ratio of absorption is X/Y, it becomes (X−x)/Y where x is smaller when a doped material is used, with the result that the rate of change is less. With a UVB sunscreen, the rate of change is also reduced as a consequence of a more stable UVB response.

The rate of loss of absorption can be determined by illuminating a sample of the composition with and without the doped TiO₂ and/or ZnO of defined thickness with UV light, as discussed in British Application No. 0315082.8.

It will be appreciated that although it will normally be the case that the bulk dopant will be the same element as the surface dopant (for simplicity of preparation), this need not necessarily be the case. (Of course, with reduced zinc oxide there is no bulk dopant.) By this means it is possible, for example, to modify the colour of the particles. Suitable dopants for the oxide particles include manganese, which is especially preferred, e.g. Mn²⁺ and Mn⁴⁺ but especially Mn³⁺, vanadium, for example V³⁺ or V⁵⁺, chromium and iron, but other metals which can be used include nickel, copper, tin, aluminium, lead, silver, zirconium, zinc, cobalt, gallium, niobium, for example Nb⁵⁺, antimony, for example Sb³⁺, tantalum, for example Ta⁵⁺, strontium, calcium, magnesium, barium, molybdenum, for example Mo³⁺, Mo⁵⁺ or Mo⁶⁺ as well as silicon. Manganese is preferably present as Mn³⁺, cobalt as Co²⁺, tin as Sn⁴⁺ as well as Mn²⁺. These metals can be incorporated singly or in combination of 2 or 3 or more. It will be appreciated that for effective bulk doping the size of the ion must be such as can readily be inserted into the crystal lattice of the particle. For this purpose Mn³⁺, vanadium, chromium and iron are generally the most effective; the ionic size of Mn²⁺ is much larger than that of Ti⁴⁺ and so there is little probability of ionic diffusion of Mn²⁺ into the TiO₂ crystal lattice. On the other hand there is no such size limitation for the elements used in surface doping; preferred surface dopants include manganese, eg. as Mn²⁺, cerium, selenium, chromium and iron as well as vanadium, typically as V⁴⁺.

The optimum total amount of the second component on, and, if present, in, the particle may be determined by routine experimentation but it is preferably low enough so that the particles are minimally coloured. Amounts as low as 0.1 mole % or less, for example 0.05 mole %, or as high as 1 mole % or above, for example 5 mole % or 10 mole %, can generally be used. Typical concentrations are from 0.5 to 2 mole % by weight. The mole ratio of dopant to host metal on the surface is typically from 2-25:98-75, usually 5-20:95-80 and especially 8-15:92-85. The amount of dopant at the surface can be determined by, for example, X-ray Photoelectron Spectroscopy (XPS).

The surface-doped particles can be obtained by any one of the standard processes for preparing such doped oxides and salts. These include techniques such as those described below. It will be appreciated that the dopant need not necessarily be present as an oxide, but may be present as a salt such as a chloride or as a salt with an oxygen-containing anion such as perchlorate or nitrate. However bulk doping techniques will generally result in some surface doping as well, and these techniques can be used in the present invention. Such techniques include a baking technique by combining particles of a host lattice (TiO₂/ZnO) with a second component in the form of a salt such as a chloride or an oxygen-containing anion such as a perchlorate or a nitrate, in solution or suspension, typically in solution in water, and then baking it, typically at a temperature of at least 300° C. Other routes which may be used to prepare the doped materials include a precipitation process of the type described in J. Mat. Sci. (1997) 36, 6001-6008 where solutions of the dopant salt and of an alkoxide of the host metal (Ti/Zn) are mixed, and the mixed solution is then heated to convert the alkoxide to the oxide. Heating is continued until a precipitate of the doped material is obtained. Further details of preparation can be found in WO 00/60994 and WO 01/40114.

It will be appreciated that such baking techniques and the like will result in dopant in the surface forming part of the crystal lattice while in other techniques the dopant will merely be adsorbed, or remain as a separate layer, on the particle surface. It may well be the case that if the dopant is to quench internally generated free radicals effectively then it needs to be in the crystal lattice.

The rutile form of titania is known to be less photoactive than the anatase form and is therefore preferred.

Doped TiO₂ or doped ZnO may be obtained by flame pyrolysis or by plasma routes where mixed metal containing precursors at the appropriate dopant level are exposed to a flame or plasma to obtain the desired product.

The zinc oxide subjected to surface doping can be reduced zinc oxide (where a skin protecting effect is desired). Reduced zinc oxide particles (i.e. particles which possess an excess of zinc ions relative to the oxygen ions) may be readily obtained by heating zinc oxide particles in a reducing atmosphere to obtain reduced zinc oxide particles which absorb UV light, especially UV light having a wavelength below 390 nm, and re-emit in the green, preferably at about 500 nm. Typically the concentration of hydrogen is from 1 to 20%, especially 5 to 15%, by volume, with the balance inert gas, especially nitrogen. A preferred reducing atmosphere is about 10% hydrogen and about 90% nitrogen by volume. The zinc oxide is heated in this atmosphere at, say, 500° to 1000° C., generally 750 to 850° C., for example about 800° C., for 5 to 60 minutes, generally 10 to 30 minutes. Typically it is heated to about 800° C. for about 20 minutes. It will be understood that the reduced zinc oxide particles will contain reduced zinc oxide consistent with minimising migration to the surface of the particles of electrons and/or positively charged holes such that when said particles are exposed to UV light in an aqueous environment the production of hydroxyl radicals is substantially reduced as discussed above.

It is believed that the reduced zinc oxide particles possess an excess of Zn²⁺ ions within the absorbing core. These are localised states and as such may exist within the band gap. A further discussion of this can be found in WO 99/60994.

The average primary particle size of the particles is generally from about 1 to 200 nm, for example about 1 to 150 nm, preferably from about 1 to 100 nm, more preferably from about 1 to 50 nm and most preferably from about 20 to 50 nm. The particle size is preferably chosen to prevent the final product from appearing coloured. Thus nanoparticles are frequently used. Since the scavenging effect is believed to be essentially catalytic it is desirable that the particles are as small as possible to maximise their surface area and hence the area of doped material on the surface. This small size has the advantage that less dopant is needed which has the consequential advantage that any colouring effect caused by the dopant is reduced. However, in one embodiment slightly larger particles for example from 100 to 500 nm, typically 100 to 400 or 450 mm especially from 150 to 300 nm and particularly 200 to 250 nm, can be employed. These provide good coverage of, for example, skin imperfections without unacceptable skin whitening.

Where particles are substantially spherical then particle size will be taken to represent the diameter. However, the invention also encompasses particles which are non-spherical and in such cases the particle size refers to the largest dimension.

The oxide particles used in the present invention may have an inorganic or organic coating. For example, the particles may be coated with oxides of elements such as aluminium, zirconium or silicon, especially silica or, for example, aluminium silicate. The particles of metal oxide may also be coated with one or more organic materials such as polyols, amines, alkanolamines, polymeric organic silicon compounds, for example, RSi[{OSi(Me)₂}xOR¹]₃ where R is C₁-C₁₀ alkyl, R¹ is methyl or ethyl and x is an integer of from 4 to 12, hydrophilic polymers such as polyacrylamide, polyacrylic acid, carboxymethyl cellulose and xanthan gum or surfactants such as, for example, TOPO. If desired the surface doping can be carried out by a coating technique either separately or in combination with the inorganic or organic coating agent. Thus for example the undoped oxide can be coated with, say, manganese oxide along with an organic or inorganic coating agent such as silica. It is generally unnecessary to coat the oxide particles to render them hydrophilic so that for the aqueous phase the particles can be uncoated. However if the particles are to be in the organic or oily phase their surface needs to be rendered hydrophobic or oil-dispersible. This can be achieved by the application directly of, for example, a suitable hydrophobic polymer or indirectly by the application of a coating, for example of an oxide such as silica (which imparts a hydrophilic property) to which a hydrophobic molecule such as a metal soap or long chain (e.g. C₁₂-C₂₂) carboxylic acid or a metal salt thereof such as stearic acid, a stearate, specifically aluminium stearate, aluminium laurate and zinc stearate.

It should be understood that the term “coating” is not to be construed as being limited to a complete covering. Indeed it is generally beneficial for the coating not to be complete since the coating can act as a barrier to the interaction of the free radicals with the dopant on or in the surface of the particle. Thus it is preferred that the coating should be discontinuous where maximum scavenging effect is desired. However it will be appreciated that dopant on the surface can still act to quench free radicals generated within the particle in which case the coating can be continuous. Since coatings of silanes and silicones which can be polymeric or short chain or monomeric silanes are generally continuous these are generally less preferred. Thus coating with an inorganic oxide is generally preferred since these generally do not result in a complete coating on the surface of the particles.

Typical coating procedures include the deposition of silica by mixing alkali such as ammonium hydroxide with an orthosilicate, such as tetraethylorthosilicate, in the presence of the particle. Alternatively the particle can first be coated with a silane such as (3-mercaptopropyl)trimethoxy silane (MPS) and then silicate e.g. sodium silicate is added. The silane attaches to the particle surface and acts as a substrate for the silicate which then polymerises to form silica. Preferably the material is extracted by freeze-drying because such a sublimation technique reduces hydrogen bond formation thereby keeping the particles small. A typical procedure is as follows:

a) 4.52 g of titania are added to 200 ml deionised water. An ultrasonic horn is used to disperse the material.

b) 1.89 ml (3-mercaptopropyl)trimethoxy silane [MPS] is added to 50 ml water under vigorous stirring

c) 20 ml of the MPS solution is added to the titania solution under vigorous stirring. This solution is stirred for two hours to allow the MPS to attach to the surface.

d) 40 ml 25% sodium silicate solution is added and the solution is stirred for one hour. The silica slowly deposits upon the MPS layer.

e) The titania is removed by centrifugation and washed three times in deionised water.

f) The material is freeze dried. A few nm layer of silica is thereby coated to the titania surface.

Similar techniques can be used for other inorganic oxides.

The compositions of the present invention can be single phase, either aqueous or oily or multiphase. Typical two-phase compositions comprise oil-in-water or water-in-oil formulations. For single phase compositions the oxide particles must of course be dispersible in that phase. Thus the particles are desirably hydrophilic if the composition is aqueous or hydrophobic if the composition is oil-based. However it may be possible to disperse untreated TiO₂ in the oily phase by appropriate mixing techniques. For two or multi-phase composition the particles must be present in the phase containing the ingredient (or one of those ingredients) to be protected. It can, though, be desirable for the particles to be present in both aqueous and oily phases even if no ingredients which are to be protected are present in one of those phases. This can cover the situation where application of the composition by the user results in some phase transfer of the ingredient(s) to be protected. Also, when an emulsion is spread on the skin it has a tendency to break down into oily and non-oily areas. When the water evaporates the oil-dispersible particles will tend to be in the oily areas thus leaving areas unprotected. This can be avoided by having both hydrophilic and hydrophobic particles in the emulsion so that some are retained in hydrophilic areas and others in hydrophobic areas. Desirably, the weight ratio of the water-dispersible particles to the oil-dispersible particles is from 1:4 to 4:1, preferably from 1:2 to 2:1 and ideally about equal weight proportions. Many organic suncreens are hydrophobic so that the particles should be hydrophobic but some organic suncreens by virtue of, in particular, acid groups are water soluble in which case the particles need to be hydrophilic in order to protect them.

The compositions of the present invention are generally for cosmetics use and may be, for example, skin tanning compositions in the form of, for example, creams, lipsticks, skin anti-ageing compositions in the form of, for example, creams, including anti-wrinkle formulations, exfoliating preparations including scrubs, creams and lotions, skin lightening compositions in the form of, for example, face creams, preparations for the hands including creams and lotions, moisturising preparations, compositions for protecting the hair such as conditioners, shampoos and hair lacquers as well as hair masks and gels, skin cleansing compositions including wipes, lotions and gels, eye shadow and blushers, skin toners and serums as well as washing products such as shower gels, bath products including bubble baths, bath oils, but, preferably, sunscreens. In this connection we should point out that the expression “cosmetic UV sunscreen composition”, as used herein, includes any composition applied to the skin which may leave a residue on the skin such as some washing products. Compositions of the present invention may be employed as any conventional formulation providing protection from UV light. The composition may also be pharmaceutical compositions suitable for topical application. Such compositions are useful, in particular, for patients suffering from disorders of the skin which are adversely affected by UV light such as those giving rise to polymorphous light eruptions.

Organic sunscreen agents which can be used in the compositions of the present invention include any conventional sunscreen agent which gives protection against UV light while if there is no other photosensitive component the sunscreen agent is photosensitive and/or is degraded by another ingredient of the composition. Suitable sunscreen agents are listed in the IARC Handbook of Cancer Prevention, vol. 5, Sunscreens, published by the International Agency for Research on Cancer, Lyon, 2001 and include:

(a) Para-aminobenzoic acids (PABA), (UVB absorbers) esters and derivatives thereof, for example amyldimethyl-; ethyldihydroxypropyl-; ethylhexyl dimethyl-; ethyl-; glyceryl-; and 4-bis-(polyethoxy)-PABA.

(b) Cinnamates (UVB) especially esters including methyl cinnamate esters and methoxycinnamate esters such as octylmethoxy cinnamate, ethyl methoxycinnamate, especially 2-ethylhexyl para-methoxycinnamate, isoamyl p-methoxy cinnamate, or a mixture thereof with diisopropyl cinnamate, 2-ethoxyethyl-4-methoxycinnamate, DEA-methoxycinnamate (diethanolamine salt of para-methoxy hydroxycinnamate) or α,β-di-(para-methoxycinnamoyl)-α′-(2-ethylhexanoyl)-glycerin, as well as diisopropyl methylcinnamate;

(c) benzophenones (UVA) such as 2,4-dihydroxy-; 2-hydroxy-4-methoxy; 2,2′-dihydroxy-4,4′-dimethoxy-; 2,2′-dihydroxy-4-methoxy-; ′2,2′,4,4′-tetrahydroxy-; and 2-hydroxy-4-methoxy-4′-methyl-benzophenones, benzenesulphonic acid and its sodium salt; sodium 2,2′-dihydroxy-4,4′-dimethoxy-5-sulphobenzophenone and oxybenzone

(d) dibenzoylmethanes (UVA) such as butyl methoxydibenzoyl methane (BMDM, referred to herein as avobenzone), especially 4-tert-butyl-4′methoxydibenzoyl methane;

(e) 2-phenylbenzimidazole-5 sulfonic acid UVB and phenyldibenzimidazole sulfonic acid and their salts;

(f) alkyl-β,β-diphenylacrylates (UVB) for example alkyl α-cyano-β,β-diphenylacrylates such as octocrylene;

(g) triazines (UVB) such as 2,4,6-trianilino-(p-carbo-2-ethyl-hexyl-1-oxy)-1,3,5 triazine as well as octyl triazone e.g. ethylhexyltriazone and diethylhexyl butamido triazone.

(h) camphor derivatives (generally UVB) such as 4-methylbenzylidene and 3-benzylidene-camphor and terephthalylidene dicamphor sulphonic acid (UVA), benzylidene camphor sulphonic acid, camphor benzalkonium methosulphate and polyacrylamidomethyl benzylidene camphor;

(i) organic pigment sunscreening agents such as methylene bisbenzotriazole tetramethyl butylphenol;

(j) silicone based sunscreening agents such as dimethicodiethyl benzal malonate.

(k) salicylates (UVB) such as dipropylene glycol-; ethylene glycol-, ethylhexyl-, isopropylbenzyl-, methyl-, phenyl-, 3,3,5-trimethyl- and TEA-salicylate (compound of 2-hydroxybenzoic acid and 2,2′2″-nitrilotris(ethanol));

(l) anthranilates (UVA) such as menthyl anthranilate as well as bisymidazylate (UVA), dialkyl trioleate (UVB), 5-methyl-2 phenylbenzoxazole (UVB) and urocanic acid (UVB).

Some compounds are effective for both UVA and UVB. These include methylene bisbenzotriazolyl tetramethylbutyl-phenol and drometrizole trisiloxane (Mexoryl XL).

The organic sunscreen agent(s) are typically present in the compositions at a concentration from 0.1 to 20%, preferably 1 to 10%, and especially 2 to 5%, by weight based on the weight of the composition.

In the compositions, the metal oxides are preferably present, in the phase or phases where they are present, at a concentration of about 0.5 to 20% by weight, preferably about 1 to 10% by weight and more preferably about 3 to 8% by weight, in particular about 4 to 7%, such as 4 to 6% for example about 5%, by weight.

The compositions may be in the form of, for example, lotions, typically with a viscosity of 4000 to 10,000 mPas, e.g. thickened lotions, gels, vesicular dispersions, creams, typically a fluid cream with a viscosity of 10,000 to 20,000 mPas or a cream of viscosity 20,000 to 100,000 mPas, milks, powders, solid sticks, and may be optionally packaged as aerosols and provided in the form of foams or sprays.

The compositions may contain any of the ingredients used in such formulations including fatty substances, organic solvents, silicones, thickeners, liquid and solid emollients, demulcents, other UVA, UVB or broad-band sunscreen agents, antifoaming agents, antioxidants such as butyl hydroxy toluene, buffers such as lactic acid with a base such as triethanolamine or sodium hydroxide, plant extracts such as Aloe vera, cornflower, witch hazel, elderflower and cucumber, activity enhancers, moisturizing agents, and humectants such as glycerol, sorbitol, 2-pyrrolidone-5-carboxylate, dibutylphthalate, gelatin and polyethylene glycol, perfumes, preservatives, such as para-hydroxy benzoate esters, surface-active agents, fillers and thickeners, sequesterants, anionic, cationic, nonionic or amphoteric polymers or mixtures thereof, propellants, alkalizing or acidifying agents, colorants and powders, including metal oxide pigments with a particle size of from 100 nm to 20000 nm such as iron oxides along with conventional (undoped) TiO₂ and ZnO.

It is known that other ingredients of cosmetic compositions, for example some surface-active agents may have the effect of degrading certain sunscreen agents in the presence of UV light. Also TiO₂ and ZnO are known to degrade certain organic sunscreens such as avobenzone as well as antioxidants such as vitamins e.g. vitamins A, B, C and E. It will be appreciated that it is particularly useful to use the doped TiO₂ and/or ZnO and/or reduced ZnO with such sunscreens. This is because TiO₂ and ZnO do generally have a positive TV absorptive effect. Thus by using the doped TiO₂ and/or ZnO and/or reduced ZnO it may be possible to use less antioxidant or make the formulation longer lasting.

The organic solvents typically comprise lower alcohols and polyols such as ethanol, isopropanol, propylene glycol, glycerin and sorbitol as well as methylene chloride, acetone, ethylene glycol monoethyl ether, diethylene glycol monobutyl ether, diethylene glycol mono-ethyl, ether, dimethyl sulphoxide, dimethyl formamide and tetrahydrofuran.

The fatty substances may comprise an oil or wax or mixture thereof, fatty acids, fatty acid esters, fatty alcohols, vaseline, paraffin, lanolin, hydrogenated lanolin or acetylated lanolin, beeswax, ozokerite wax and paraffin wax.

The oils typically comprise animal, vegetable, mineral or synthetic oils and especially hydrogenated palm oil, hydrogenated castor oil, vaseline oil, paraffin oil, Purcellin oil, silicone oil such as polydimethyl siloxanes and isoparaffin.

The waxes typically comprise animal, fossil, vegetable, mineral or synthetic waxes. Such waxes include beeswax, Carnauba, Candelilla, sugar cane or Japan waxes, ozokerites, Montan wax, microcrystalline waxes, paraffins or silicone waxes and resins.

The fatty acid esters are, for example, isopropyl myristate, isopropyl adipate, isopropyl palmitate, octyl palmitate, C₁₂-C₁₅ fatty alcohol benzoates (“FINSOLV TN” from FINETEX), oxypropylenated myristic alcohol containing 3 moles of propylene oxide (“WITCONOL APM” from WITCO), capric and caprylic acid triglycerides (“MIGLYOL 812” from HULS).

The compositions may also contain thickeners such as cross-linked or non cross-linked acrylic acid polymers, and particularly polyacrylic acids which are cross-linked using a polyfunctional agent, such as the products sold under the name “CARBOPOL” by the company GOODRICH, cellulose, derivatives such as methylcellulose, hydroxymethylcellulose, hydroxypropyl methylcellulose, sodium salts of carboxymethyl cellulose, or mixtures of cetylstearyl alcohol and oxyethylenated cetylstearyl alcohol containing 33 moles of ethylene oxide.

Suitable emollients include stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rapeseed oil, safflower seed oil, evening primrose oil, soybean oil, sunflower seed oil, avocado oil, sesame seed oil, coconut oil, arachis oil, caster oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate.

Suitable propellants include propane, butane, isobutane, dimethyl ether, carbon dioxide, nitrous oxide.

Suitable powders include chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate.

When the compositions of the present invention are sunscreens they may be in the form of, for example, suspensions or dispersions in solvents or fatty substances or as emulsions such as creams or milks, in the form of ointments, gels, solid sticks or aerosol foams. The emulsions, which can be oil-in-water or water-in-oil emulsions, may further contain an emulsifier including anionic, nonionic, cationic or amphoteric surface-active agents; for a water-in-oil emulsion the HLB is typically from 1 to 6 while a larger value i.e >6 is desirable for an oil-in-water emulsion. Generally water amounts to up to 80%, typically 5 to 80%, by volume. Specific emulsifiers which can be used include sorbitan trioleate, sorbitan tristearate, glycerol monooleate, glycerol monostearate, glycerol monolaurate, sorbitan sesquioleate, sorbitan monooleate, sorbitan monostearate, polyoxyethylene (2) stearyl ether, polyoxyethylene sorbitol beeswax derivative, PEG 200 dilaurate, sorbitan monopalmitate, polyoxyethylene (3.5) nonyl phenol, PEG 200 monostearate, sorbitan monostearate, sorbitan monolaurate, PEG 400 dioleate, polyoxyethylene (5) monostearate, polyoxyethylene (4) sorbitan monostearate, polyoxyethylene (4) lauryl ether, polyoxyethylene (5) sorbitan monooleate, PEG 300 monooleate, polyoxyethylene (20) sorbitan tristearate, polyoxyethylene (20) sorbitan trioleate, polyoxyethylene (8) monostearate, PEG 400 monooleate, PEG 400 monostearate, polyoxyethylene (10) monooleate, polyoxyethylene (10) stearyl ether, polyoxyethylene (10) cetyl ether, polyoxyethylene (9.3) octyl phenol, polyoxyethylene (4) sorbitan monolaurate, PEG 600 monooleate, PEG 1000 dilaurate, polyoxyethylene sorbitol lanolin derivative, polyoxyethylene (12) lauryl ether, PEG 1500 dioleate, polyoxyethylene (14) laurate, polyoxyethylene (20) sorbitan monostearate, polyoxyethylene (20) sorbitan monooleate, polyoxyethylene (20) stearyl ether, polyoxyethylene (20) sorbitan monopalmitate, polyoxyethylene (20) cetyl ether, polyoxyethylene (25) oxypropylene monostearate, polyoxyethylene (20) sorbitol monolaurate, polyoxyethylene (23) lauryl ether, polyoxyethylene (50) monostearate, and PEG 4000 monostearate. Alternatively the emulsifier can be silicone surfactant, especially a dimethyl polysiloxane with polyoxyethylene and/or polyoxypropylene side chains, typically with a molecular weight of 10,000 to 50,000, especially cyclo-methicone and dimethicone copolyol. They may also be provided in the form of vesicular dispersions of ionic or nonionic amphiphilic lipids prepared according to known processes.

The following Example, in addition to Example 1 given above in respect of the first embodiment, further illustrates the embodiment of the present invention.

### EXAMPLE 2

A comparison was made between formulations differing solely in the nature of the TiO₂ incorporated.

### Preparation of Sunscreen Formulations

The sunscreen formulations were based on a procedure by Stanley Black (www.sblack.com Formula Reference 1629).

| | % w/w |
|---|---|
| Phase A | |
| Water | 80.35 |
| Propylene Glycol | 2.00 |
| Methylparaben | 0.15 |
| Aloe Vera Gel x1 | 0.10 |
| Phase B | |
| Lexemul 561 (Glyceryl Stearate, PEG-100 | 5.00 |
| Stearate) | |
| Lexemul GDL (Glyceryl Dilaurate) | 1.50 |
| Stearyl Alcohol NF | 0.30 |
| Lexol IPM (Isopropyl Myristate) | 1.00 |
| Lexol EHP (Octyl Palmitate) | 2.00 |
| Dow Corning 200 Fluid 200 cs (Dimethicone) | 0.50 |
| Propylparaben | 0.10 |
| Parsol 1789 (BMDM) | 2.00 |
| Titanium Dioxide | 5.00 |

The formulations were produced as follows:
Heat phase A to 75° C.
Heat phase B to 75° C.
Add phase A to phase B with vigorous stirring.
Cool to room temperature with stirring.

The TiO₂ used was as follows:
A. TiO₂ doped with manganese to a level of approximately 1 mole %; primary particle size 20-30 nm; crystal form 99% rutile; no coating.
B. Uvinul TiO₂ from BASF
Primary particle size—c.21 nm
Crystal form—75% Anatase/25% Rutile
Coating—Trimethyylcaprylylsilane at 5%
MT100AQ from Tayca Corp
Primary particle size—15 nm
Crystal form—c.100% Rutile
Coating—Alumina/silica/alginic acid at up to 30%

The formulations were tested using the DPPH assay technique of Example 1, on artificial skin and using a cuvette and absorbance measurements taken.

### Artificial Skin.

Vitro Skin was obtained from IMS Testing Group. The Vitro-Skin was cut into 6.2×9 cm rectangles and placed in a closed, controlled-humidity chamber containing 15% glycerin overnight. Sunscreen samples (formulations) were placed on the re-hydrated films at a loading of 2 mg/ml and spread evenly using a latex covered finger. The film was mounted into a 6×6 cm glassless slide mount and left to dry for 15 minutes. UV absorbance was measured and then the sample illuminated by a xenon arc solar simulator for 2 hours. Absorbance measurements were recorded following 5, 15, 30, 60, 90 and 120 minutes illumination.

### Cuvette

Samples were loaded into a 10 μm cuvette (approx. sample volume of 4 μl i.e. liquid). UV absorbance was measured pre-illumination and also following 5, 15, 30, 60, 90 and 120 minutes illumination by a xenon arc solar simulator or a SOL2 solar simulator (Honle U technology). A comparison with Nivea SPF10 was also made.

For DPPH assay, the formulations contained 2% Parsol 1789 (avobenzone).

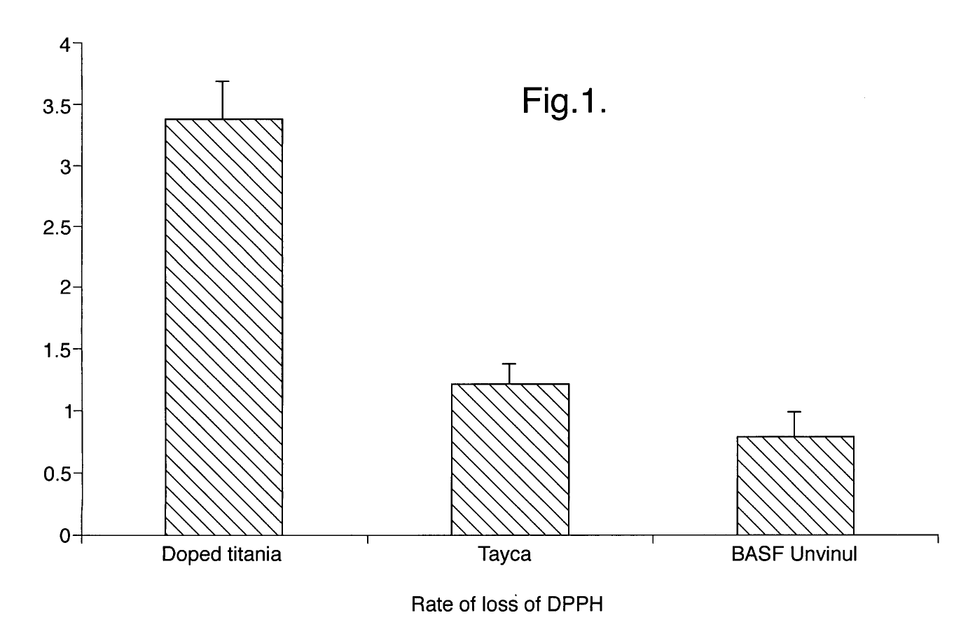

The results obtained are shown in FIG. 1. Clearly the scavenging activity of the doped TiO2 is significantly superior to that of the commercial products, the rate of loss of DPPH being around 3 time greater.

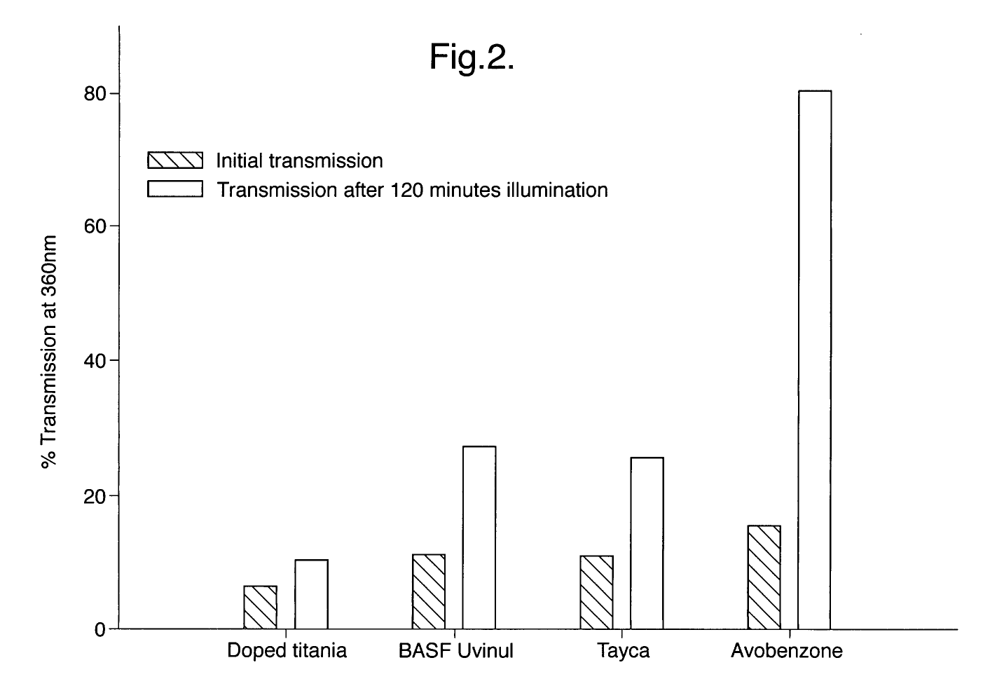

FIG. 2 gives the results of light transmission at 360 nm at time 0 and at time 120 minutes for formulations containing 2% avobenzone on hydrated artificial skin.

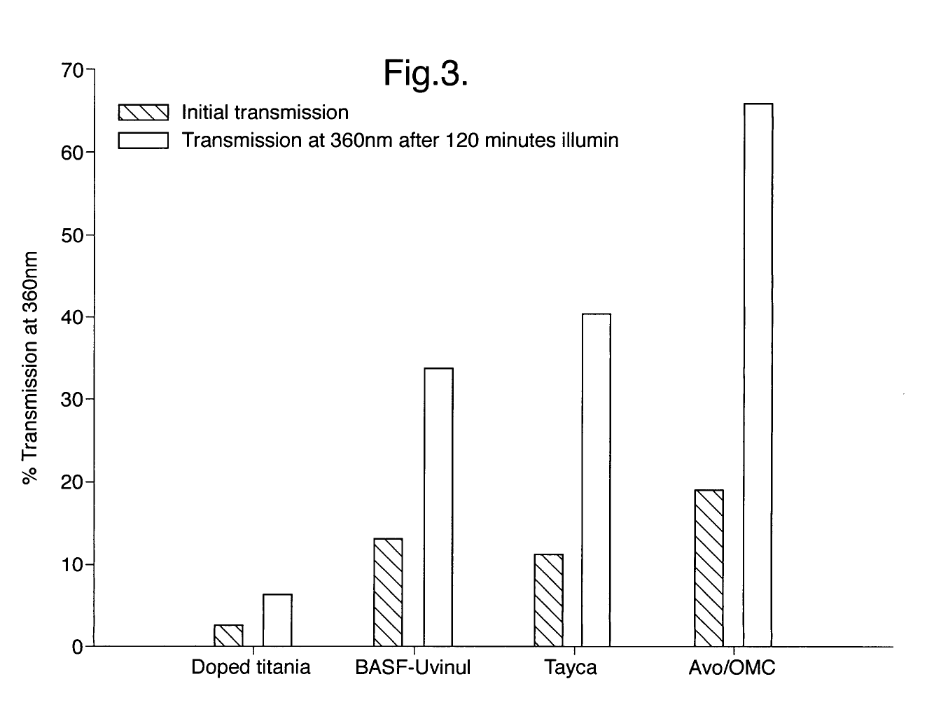

FIG. 3 gives the results of light transmission at 360 nm for formulations containing 2% avobenzone (AVO) and 5% octylmethoxycinnamate (OMC) on hydrated artificial skin.

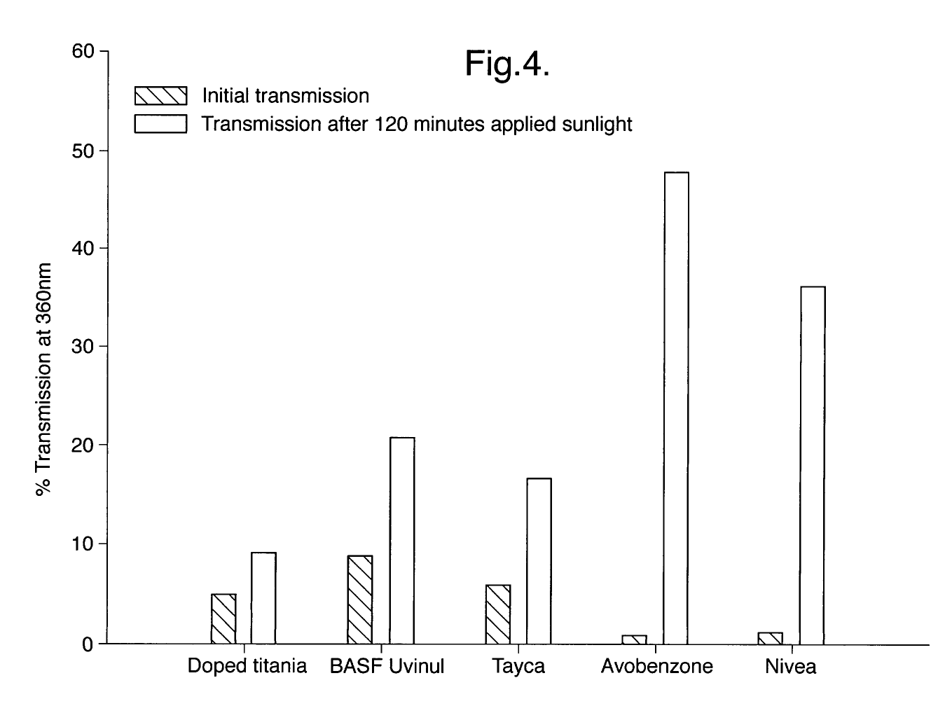

FIG. 4 gives the results of light transmission at 360 nm at time 0 and at time 120 minutes for formulations containing 2% avobenzone in the cuvette

These results clearly show the superiority of the doped TiO₂ in reducing UVA transmission. The fact that the ratios of the values at time 0 and time 120 minutes are significantly different implies that a reduction in free radical load, from both reduced generation and scavenging, is present in the formulations where doped titania is used.

### The Third Embodiment

The third embodiment of the present invention relates to polymeric compositions for a variety of uses.

It is well known that many polymeric compositions are adversely affected by light, in particular UV light. This can result in a variety of physical properties of the composition being affected. Typically, solid plastics compositions have their strength adversely affected so that, over time, they become more brittle. Similar comments apply to coating compositions. Other properties which can be adversely affected include colour. It is well known, for example, that coating compositions such as paints are adversely affected by light so that fading or, in the case of white formulations, yellowing occurs.

Various attempts have been made to counteract these adverse effects. This has included incorporating light stabilisers into the composition, typically hindered amines. However, incorporation of such light stabilisers is relatively expensive and not always particularly effective.

The present invention resides in the discovery that the incorporation of particular types of titanium dioxide and zinc oxide can effectively counteract the adverse effect of exposure to light, typically sun light.

In our GB Application No. 0310365.2 we disclose that the degradation of polymeric compositions can be retarded if the compositions also have present either zinc oxide or titanium dioxide which has been doped with a second element or reduced zinc oxide. In other words by using these doped materials or reduced zinc oxide rather than ordinary titanium dioxide or zinc oxide it is, for example, possible either to provide a polymeric composition which gives better protection against UV light or a composition having the same resistance to degradation but containing a smaller quantity of light stabiliser. The application thus describes a polymeric composition which comprises an amount of one or more organic or inorganic components which are photosensitive and/or which are degraded by another ingredient of the composition, and an amount of either TiO₂ and/or ZnO which has been doped with a second element or reduced ZnO, this composition having a rate of deterioration of a UV light-sensitive physical factor at least 5% less than that of a composition having the same formulation except that it does not contain the TiO₂ and/or ZnO which has been doped with a second element or reduced ZnO.

By a “physical factor” is meant a measurable value of a physical property of the composition which is adversely affected by UV light. Examples of such physical factors include degradation and, in consequence, strength, colour change (e.g. for paints and textiles) and photographic stability (e.g. for photographic films).

Thus if the rate of deterioration of a physical factor is X then the amount of the component(s) which are photosensitive and/or which are degraded by another ingredient of the composition, possesses a said rate of deterioration of Y where Y is greater than X by at least 5%, and the amount of doped TiO₂ and/or ZnO and/or reduced ZnO reduces the said rate of loss from Y to X. The present invention also provides the use of a doped TiO₂/ZnO and/or reduced ZnO to reduce the concentration of one or more light stabilisers in a polymeric composition as well as to reduce the rate of deterioration of a physical factor of a polymeric composition. The present invention further provides a method of improving the stability of a physical factor of a composition which comprises one or more components which are photosensitive and/or which are degraded by another ingredient of the composition which comprises incorporating into the composition a doped TiO₂/ZnO and/or reduced ZnO.

As mentioned in connection with the second embodiment, it is important that if the oxide is to be really effective there must be dopant on its surface which can interact with the component of the composition to be protected. Although existing methods for doping in the bulk will normally also result in some dopant in or on the surface of the particle, it is possible according to the present invention to use materials which are only surface doped i.e. where there is dopant only in or on the surface of the particle. In one embodiment such materials may be used in a single phase formulation

Accordingly the present invention provides (although not dependant on the above theory) a composition which comprises an amount of one or more organic or inorganic components which are photosensitive and/or which are degraded by another ingredient of the composition and an amount of TiO₂ and/or ZnO which has been doped at least on or in a surface thereof with one or more other elements, typically with one i.e with only a second element.

The composition may be polymeric, which as used herein means that the composition may comprise one or more polymeric materials, typically constituting at least 1%, preferably 5% by weight of the composition. Also, the composition may be solid or liquid. Where a polymeric material is present it may comprise at least part of the organic component and/or it may comprise a binder and/or other component of the composition.

Where the particle has been bulk doped there will, in general, be dopant throughout the particle. On the other hand where the particle has been “surface doped” (i.e. the dopant is only in or on the surface) there will be a concentration gradient e.g. such that the ratio of dopant atoms to titanium or zinc atoms at the surface or outmost “skin” of the particle is greater than the ratio in the core or centre where it may be zero. In general, the composition has a formulation which has a rate of deterioration of a UV light-sensitive physical factor at least 5% less than that of a composition having the same formulation except that it does not contain the said TiO₂ and/or ZnO which has been doped with a second element.

By “a polymeric composition” as used herein is meant a composition which comprises one or more polymeric materials. The composition can be solid or liquid.

In some instances, the composition of the present invention will contain TiO₂ and/or ZnO which has not been doped. Typically such undoped TiO₂/ZnO will be present as pigment, generally having a particle size of at least 100 nm.

Typical solid materials include polymeric solids including three dimensional objects, films and fibres as well as textiles and fabrics e.g. clothing and netting made from woven and non-woven fibres as well as foamed articles; solids which are not fibres are sometimes preferred. Three-dimensional objects include those made by melt-forming processes including extruded and moulded articles. Typical articles to which the present invention may be applied include generally external household and building materials including blinds and plastics curtains, trellis, pipes and guttering, cladding and facings such as soffit board and plastics roofing material which can be profiled as with corrugated sheeting, doors and windows frames. Other articles include advertising hoardings and the like e.g. advertising boards on vehicle sides as well as vehicle bodies and body parts including bumpers for cars, buses and trucks as well as roofs which can be used also for boats, as well as superstructures and hulls for boats and also bodies for lawnmowers and tractors and yachts, along with containers such as bottles, cans, drums, buckets and oil and water storage containers. Other objects include garden furniture. In one embodiment the solids are not transparent.

Films to which the present invention can be applied include self supporting as well as non-self supporting films such as coatings. Self-supporting films to which the present invention applies include photographic films, packaging film and plastics film bearing indicia, typically as advertising film, which can also be applied over advertising hoardings. Such films can contain one or more customary ingredients for such products. Thus photographic film will contain one or more dyes or dye couplers and, optionally, a silver halide.

In some instances the polymeric composition itself is not liable to degradation but the composition is intended to protect a substrate or, in the case of a container, something placed in it. Thus such compositions can contain the doped TiO₂/ZnO. Examples include pigmented and non-pigmented containers, typically bottles.

Accordingly, the present invention also provides a self-supporting polymer composition, or a varnish composition, intended to protect a composition adjacent thereto from the adverse effects of light, and which comprises TiO₂ and/or ZnO which has been surface doped with at least a second element. In one embodiment the composition is 3-dimensional and comprises a surface layer with the TiO₂ and/or ZnO while the non-surface part is generally not wood or a reconstituted wood such as chipboard, plywood or fibreboard and is preferably synthetic.

Coating compositions are typically paints and varnishes which contain a polymer either as the active ingredient as in some varnishes or as a support as in paints along with furniture polishes, waxes and creams; they can be aqueous or non aqueous i.e. contain an organic solvent in which case they can be mono-phase or poly-phase, typically as an oil-in-water or water-in-oil emulsion. This coating composition can be in the form of a waterproofing agent. These coating compositions can contain one or more customary ingredients for such products. Some cosmetics compositions contain one or more polymers; such compositions are less preferred in the present invention.

The polymers which can be used in the compositions of the present invention include natural and synthetic polymers which may be thermoplastic or thermosetting.

The suitable polymers which may be homopolymers or copolymers which can be random, block or graft copolymers; the polymers can be crosslinked. Such polymers may be saturated or unsaturated. Typical polymers include alkylene polymers such as ethylene and propylene polymers, typically homopolymers, including polyethylene foams, siloxane and sulphide polymers, polyamides such as nylon, polyesters, such as PET, acrylate and methacrylate polymers e.g. poly(methyl methacrylate), polyurethanes, including foams, vinyl polymers such as styrene polymers e.g. ABS, including polystyrene foam, vinyl chloride polymers and polyvinyl alcohol as well as engineering thermoplastics including aromatic polymers, e.g. polymers such as linear aromatic semi-crystalline polymers such as PEEK and PES. Fluorinated polymers such as PTFE and polyvinylidene fluoride can be used. The polymers can be thermosetting as with epoxy resins as well as phenolic, urea, melamine and polyester resins

Natural polymers which can be used include cellulosic polymers, as in paper including starch, polysaccharides, lignins, and polyisoprenes such as natural rubbers.

It will be appreciated that some polymers can be regarded as photostable in that there is no, or no significant, change in physical characteristics on exposure to UV light. These polymers are, therefore, not photosensitive and their use does not fall within the scope of the present invention.

Typical polymers for different applications include the following: (a) polyester, polyamide e.g. nylon, acrylics for fibres and fabrics; (b) polyester, polyvinyl chloride, polyethylene, polypropylene for bottles and the like; (c) polyethylene, polypropylene, polyvinyl chloride for film (non active such as packaging).

The compositions can contain the usual additional ingredients characteristic for the composition in question including inorganic and organic pigments, including “ordinary” TiO₂ and/or ZnO, fillers and extenders as well as light stabilisers, typically hindered amine stabilisers. The additional ingredients may themselves be susceptible to attack, with the degraded components potentially causing degradation of the polymer or other component of the composition.

The rate of colour change can be determined by illuminating a sample of the composition with and without the doped TiO₂ or ZnO with sunlight or visible light and measuring the spectral response of the composition over a given period and determining the change in wavelength emitted. Accelerated ageing tests using, for example a Fadeometer, can be used for this purpose.

The rate of loss of strength of an article of the present invention can be determined in a similar manner by measuring tensile properties such as elongation at break or Young's modulus, using standard equipment such as an Instron tester; again an accelerated ageing procedure is beneficial.

While any reduction in the wavelength change or other physical factor is an advantage, it is generally desirable that the presence of the doped oxide should reduce the rate of change by an amount of at least 5%, preferably at least 10%, more preferably at least 15%, especially at least 20% and most preferably at least 40%.

It will be appreciated that although it will normally be the case that the bulk dopant will be the same element as the or each surface dopant (for simplicity of preparation), this need not necessarily be the case. (Of course with reduced zinc oxide there is no bulk dopant.) By this means it is possible, for example, to modify the colour of the particles. Suitable dopants for the oxide particles include manganese, which is especially preferred, e.g. Mn²⁺ but also Mn³⁺, vanadium, for example V³⁺ or V⁵⁺, chromium and iron but other metals which can be used include nickel, copper, tin, especially Sn⁴⁺, aluminium, lead, silver, zirconium, zinc, cobalt, especially Co²⁺, gallium, niobium, for example Nb⁵⁺, antimony, for example Sb³⁺, tantalum, for example Ta⁵⁺, strontium, calcium, magnesium, barium, molybdenum, for example Mo³⁺, Mo⁵⁺ or Mo⁶⁺ as well as silicon. These metals can be incorporated singly or in combinations of two or three or more. It will be appreciated that for effective bulk doping the size of the ion must be such as can readily be inserted into the crystal lattice of the particle. For this purpose Mn³⁺, vanadium, chromium and iron are generally the most effective; the ionic size of Mn²⁺ is much larger than that of Ti⁴⁺ and so there is little probability of ionic diffusion of Mn²⁺ into the TiO₂ crystal lattice. On the other hand there is no such size limitation for the elements used in surface doping; preferred surface dopants include manganese, eg. as Mn²⁺, cerium, selenium, chromium and iron.

The optimum total amount of the second component on, and, if present, in, the particle may be determined by routine experimentation but it is preferably low enough so that the particles are minimally coloured. Amounts as low as 0.1 mole % or less, for example 0.05 mole %, or as high as 1 mole % or above, for example 5 mole % or 10 mole %, can generally be used. Typical concentrations are from 0.5 to 2 mole % by weight. The mole ratio of dopant to host metal on the surface is typically from 2-25:98-75, usually 5-20:95-80 and especially 8-15:92-85. The amount of dopant at the surface can be determined by, for example, X-ray Photoelectron Spectroscopy (XPS).

The surface-doped particles can be obtained by any one of the standard processes for preparing such doped oxides and salts. These include techniques such as those described below. It will be appreciated that the dopant need not necessarily be present as an oxide but as a salt such as a chloride or a salt of an oxygen-containing anion such as perchlorate or nitrate. However bulk doping techniques will generally result in some surface doping as well and these techniques can be used in the present invention. Such techniques include a baking technique by combining particles of a host lattice (TiO₂/ZnO) with a second component in the form of a salt such as a chloride or an oxygen-containing anion such as a perchlorate or a nitrate, in solution or suspension, typically in solution in water, and then baking it, typically at a temperature of at least 300° C. Other routes which may be used to prepare the doped materials include a precipitation process of the type described in J. Mat. Sci. (1997) 36, 6001-6008 where solutions of the dopant salt and of an alkoxide of the host metal (Ti/Zn) are mixed, and the mixed solution is then heated to convert the alkoxide to the oxide. Heating is continued until a precipitate of the doped material is obtained. Further details of preparation can be found in WO 00/60994 and WO 01/40114.

It will be appreciated that such baking techniques and the like will result in dopant in the surface forming part of the crystal lattice, while in other techniques the dopant will merely be adsorbed, or remain as a separate layer, on the particle surface. It is thought likely that if the dopant is to quench internally generated free radicals then it needs to be in the crystal lattice.

The rutile form of titania is known to be less photoactive than the anatase form and is therefore preferred. Zinc oxide can be in the form of reduced zinc oxide particles (i.e. particles which possess an excess of zinc ions relative to the oxygen ions).

Doped TiO₂ or doped ZnO may be obtained by flame pyrolysis or by plasma routes where mixed metal containing precursors at the appropriate dopant level are exposed to a flame or plasma to obtain the desired product.

Further details of such particles can be found in WO 99/60994.

The average primary particle size of the particles is generally from about 1 to 200 nm, for example about 1 to 150 nm, preferably from about 1 to 100 nm, more preferably from about 1 to 50 nm and most preferably from about 20 to 50 nm. Since the scavenging effect is believed to be essentially catalytic it is desirable that the particles are as small as possible to maximise their surface area and hence the area of doped material on the surface. This small size has the advantage that less dopant is needed, which has the consequential advantage that any colouring effect caused by the dopant is reduced.

Where particles are substantially spherical then particle size will be taken to represent the diameter. However, the invention also encompasses particles which are non-spherical and in such cases the particle size refers to the largest dimension.

The oxide particles used in the present invention may have an inorganic or organic coating. For example, the particles may be coated with oxides of elements such as aluminium, zirconium or silicon, especially silica or, for example, aluminium silicate. The particles of metal oxide may also be coated with one or more organic materials such as polyols, amines, alkanolamines, polymeric organic silicon compounds, for example, RSi[{OSi(Me)₂}xOR¹]₃ where R is C₁-C₁₀ alkyl, R¹ is methyl or ethyl and x is an integer of from 4 to 12, hydrophilic polymers such as polyacrylamide, polyacrylic acid, carboxymethyl cellulose and xanthan gum or surfactants such as, for example, TOPO. If desired the surface doping can be carried out by a coating technique either separately or in combination with the inorganic or organic coating agent. Thus for example the undoped oxide can be coated with, say, manganese oxide along with an organic or inorganic coating agent such as silica. It is generally unnecessary to coat the oxide particles to render them hydrophilic so that for the aqueous phase the particles can be uncoated. However if the particles are to be in the organic or oily phase their surface needs to be rendered hydrophobic or oil-dispersible. This can be achieved by the application directly of, for example, a suitable hydrophobic polymer or indirectly by the application of a coating, for example of an oxide such as silica (which imparts a hydrophilic property) to which a hydrophobic molecule such as a metal soap or long chain (e.g. C₁₂-C₂₂) carboxylic acid or a metal salt thereof such as stearic acid, a stearate, specifically aluminium stearate, aluminium laurate and zinc stearate.

It should be understood that the term “coating” is not to be construed as being limited to a complete covering. Indeed it is generally beneficial for the coating not to be complete since the coating can act as a barrier to the interaction of the free radicals with the dopant on or in the surface of the particle. Thus it is preferred that the coating should be discontinuous where maximum scavenging effect is desired. However it will be appreciated that dopant on the surface can still act to quench free radicals generated within the particle in which case the coating can be continuous. Since coatings of silanes and silicones which can be polymeric or short chain or monomeric silanes are generally continuous these are generally less preferred. Thus coating with an inorganic oxide is generally preferred since these generally do not result in a complete coating on the surface of the particles.

Typical coating procedures include the deposition of silica by mixing alkali such as ammonium hydroxide with an orthosilicate, such as tetraethylorthosilicate, in the presence of the particle. Alternatively the particle can first be coated with a silane such as (3-mercaptopropyl)trimethoxy silane (MPS) and then silicate e.g. sodium silicate is added. The silane attaches to the particle surface and acts as a substrate for the silicate which then polymerises to form silica. Similar techniques can be used for other inorganic oxides.

The compositions of the present invention can be single phase, either aqueous (or oily or generally hydrophobic) or multiphase. Typical two-phase compositions comprise oil-in-water or water-in-oil formulations. For single phase compositions the oxide particles must of course be dispersible in that phase. Thus the particles are desirably hydrophilic if the composition is aqueous or hydrophobic if the composition is oil-based. However it may be possible to disperse untreated TiO₂ in the oily phase by appropriate mixing techniques. For two or multi-phase composition the particles must be present in the phase containing the ingredient (or one of those ingredients) to be protected. It can, though, be desirable for the particles to be present in both aqueous (or generally hydrophilic) and oily (or generally hydrophobic) phases even if no ingredients which are to be protected are present in one of those phases. Desirably, the weight ratio of the water-dispersible particles to the oil-dispersible particles is from 1:4 to 4:1, preferably from 1:2 to 2:1 and ideally about equal weight proportions.

In the compositions the metal oxides are preferably present at a concentration of about 0.5 to 20% by weight, preferably about 1 to 10% by weight and more preferably about 3 to 8% by weight.

The following Example, in addition to Example 1 given above in respect of the first embodiment, further illustrates the present invention.

### EXAMPLE 3

### Preparation of doped TiO₂

### Coprecipitation Method

Distilled water (170 cm³), conc. HCl (12 cm³) and propan-2-ol (12 cm³) were mixed together at room temperature with stirring. The appropriate metal salt at the calculated percentage loading was added to the solution (1% loading in this case). After thorough mixing, titanium isopropoxide (10.4 cm³) was gradually added using a pipette. A gelatinous precipitate was formed instantly. After the solution became clear it was heated in a water bath. The water bath temperature was slowly increased from room temperature to 328 K over a period of a few hours. The solutions were left overnight. The resulting precipitate was decanted and dried at 353 K and then placed in an oven for a few hours at 373 K. The samples were then calcined at either 873 K, initially, and then at 1273 K (to ensure formation of rutile crystals) in air for 3 h. (heating regime 298 Kelvin to the chosen temperature at 200 Kelvin/h, dwell time=3 h followed by cooling to 298 K at 200 K/h).

### Absorption Method

The appropriate metal salt (1% loading) was dissolved in methanol along with TiO₂ powder Degussa P25 (0.05 moles ˜75% anatase and 25% rutile; surface area ˜50 m²/_{g}; average particle size ˜30 nm). The solution was stirred for a few hours and then the solvent was evaporated to leave TiO₂ powder. The powder was placed in an oven at 423 K for 2-3 h and later calcined in air at 873 K using the same heating regime as for the co-precipitation method.

EPR Electron paramagnetic resonance was carried out at low temperatures (100 K) at the EPSRC EPR facility at Cardiff University.

### Mn-Doped TiO_{2,} Coprecipitation Method

Mn(II)-doped TiO₂ samples were prepared via both preparation methods and their EPR spectra obtained. The spectrum of 1% Mn(II)-doped TiO₂, made by the coprecipitation route, shows Mn⁴⁺ occupying a substitutional site and Mn²⁺ occupying an interstitial site.

### Mn-Doped TiO₂, Absorption Method

The spectrum of 1% Mn(II)-doped TiO₂, made by the absorption method, shows substitutionally incorporated Mn⁴⁺ and Mn²⁺ substitutionally incorporated. Also there is evidence to suggest surfacial Mn²⁺.

### V(IV) Doped TiO₂

V(IV)-doped TiO₂ samples were prepared via both preparation methods and their EPR spectra obtained. The spectrum of 1% V(IV)-doped TiO₂, made by the absorption route, shows a poorly resolved spectrum which is due to V⁴⁺ ions superimposed on a broad resonance which is probably due to Ti³⁺ ions. The spectrum of 1% V(IV)-doped TiO₂, made by the coprecipitation method, shows a well-resolved spectrum of an eightfold hyperfine line resonances due to interaction between magnetic moments of the ⁵¹V nucleus with paramagnetic V⁴⁺ ions which is due to V⁴⁺ occupying substitutional sites in the TiO₂ matrix.

### V(V) Doped TiO₂

V(V)-doped TiO₂ samples were prepared via both methods and their EPR spectra obtained. V(V)-doped TiO₂ samples prepared via the coprecipitation show that V⁴⁺ is occupying a substitutional site, whereas the V(V)-doped TiO₂, produced by the absorption method, showed poorly resolved spectra reflecting the possibility that the vanadium ions are not substituting into the TiO₂ lattice but exist on the surface.

### Preparation of PVC Films

Poly(vinyl chloride) (1 g) was dissolved in HPLC grade tetrahydrofuran (20 cm³) and the corresponding amount of modified TiO₂ pigment added (4% loading in this case). The solution was then sonicated/stirred for approximately 1 h. Thin films (100-150 μm) were prepared by pouring the solution into disposable aluminium trays (area=8.55 cm²) and allowing the solvent to evaporate. The weight of the resulting disc was then obtained (four decimal point balance) and recorded. From these data the thickness could be obtained by using the known area, weight and density of the PVC film. The thickness was then verified by analysing the film under an Olympus BH2 scanning optical microscope. The IR spectra were recorded and samples chosen for size according to their relative absorbances at 2913 cm⁻¹. The films were then irradiated in a QUV weatherometer (Q Panel Company) equipped with 8 UV_{B} 300 W bulbs at a temperature of 318 K.

### UV Irradiation Equipment

A Q Panel QUV accelerated weatherometer was used. The device is essentially an UV irradiation tank. 8 fluorescent bulbs (300 W), selected as UV_{B} wavelength, are fitted inside the apparatus and a moisture bath can also be used to force harsh conditions. Thin film samples are mounted onto the plates and placed on the sides of the instrument. The light intensity delivered within the QUV weatherometer was determined using the potassium ferrioxalate system. The intensity at the side of the instrument was calculated to be 1.82×10¹⁷ quanta/s.

### Results

IR absorption spectra were recorded using a Perkin-Elmer 1000 spectrophotometer (range 3200 cm⁻¹-400 cm⁻¹). Resolution was predetermined at 4 cm⁻¹. The appearance of a carbonyl peak at 1718 cm⁻¹ was monitored and calculated. The appearance of this peak over time was recorded and normalised with respect to the CH band at 2913 cm⁻¹ to produce the “carbonyl index”.

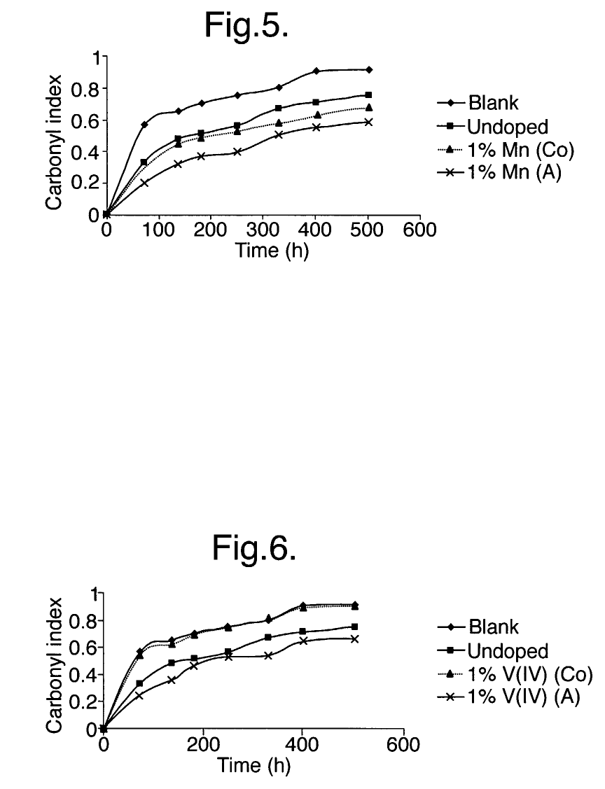

The results for the effect of addition of Mn and V to TiO₂ upon the photodegradation of PVC film is shown in FIGS. 5 to 7. “1% Mn (Co)” is Mn-doped TiO₂ made by the coprecipitation method and “1% Mn (A)” is made by the absorption method; similar comments apply to V-doped materials. The protection factor was calculated after 500 h by comparison of the carbonyl indices of the doped samples with that of the undoped sample.

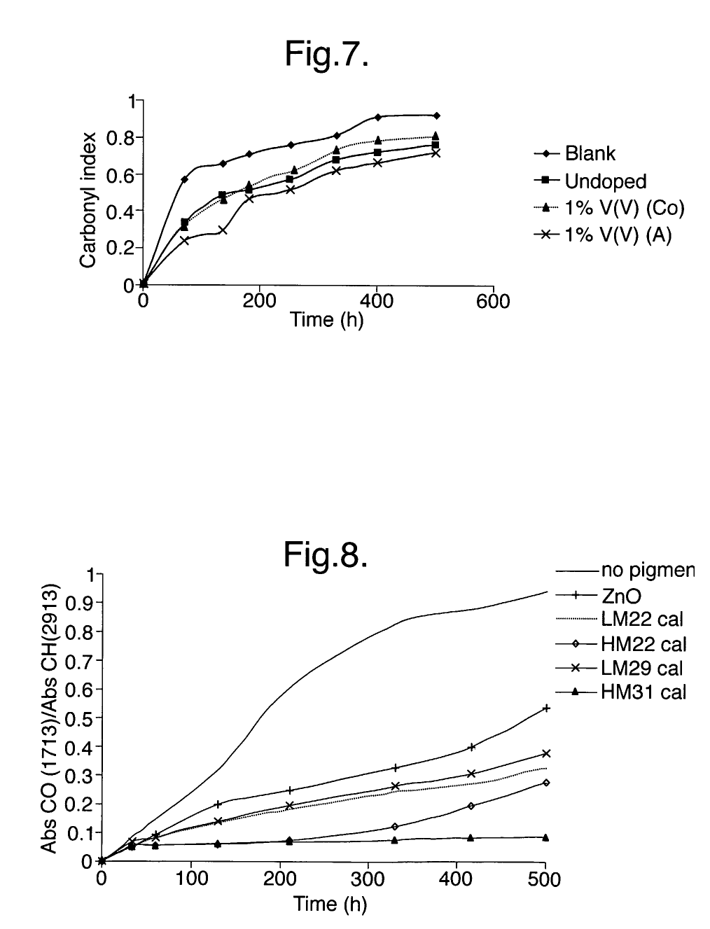

In FIG. 5, the 1% Mn (coprecipitation method) sample is ˜9% more effective than the undoped TiO₂ at protecting the PVC film whereas the 1% Mn (absorption method) is ˜23% more effective. In FIG. 6 the 1% V (coprecipitation method) sample is ˜20% less effective than the undoped TiO₂ at protecting the PVC film whereas the 1% V (absorption method) is ˜12% more effective. In FIG. 7 the 1% V (coprecipitation method) sample is ˜6% less effective than the undoped TiO₂ at protecting the PVC film whereas the 1% V (absorption method) is ˜6% more effective.

FIG. 8 shows the effect of adding Mn doped ZnO (calcined at 573 k) to PVC films. “LM” and “HM” refer to low and high concentration of Mn. “HM31 cal” shows a 27% improvement in PVC film protection compared to undoped ZnO. All of the doped materials show more protection than the undoped reference.

### The Fourth Embodiment

This relates to compositions suitable for use in agriculture, horticulture and veterinary medicine.

It is well known that many of the active ingredients of veterinary, agricultural and horticultural compositions such as herbicides and insecticides are adversely affected by UV light. Such organic compounds have a tendency to degrade or decompose under the influence of UV light either to inactive compounds or compounds which have an adverse effect upon the area being treated. As a result it is necessary to store these products in special containers which do not allow the penetration of UV light. Otherwise the shelf life of the product is too short.

In our GB Application No. 0312703.2 referred to above, we disclose that the adverse effects of UV light on such organic compounds can be reduced and/or eliminated by incorporating in the composition titanium dioxide and/or zinc oxide which has been doped with a second element and/or reduced zinc oxide. In other words by incorporating this specific oxide in the formulation it is possible to dispense with the use of special containers and/or extend the life of the product. In addition its presence enables the user to use less of the product. The application thus describes a composition suitable for veterinary, agricultural or horticultural use which comprises at least one organic veterinarally, agriculturally and/or horticulturally active compound, and titanium dioxide and/or zinc oxide which has been doped with a second element and/or reduced zinc oxide as well as a method for treating a veterinary, agricultural or horticultural species at a locus which comprises treating the locus with such a composition.

While any reduction in the loss of TV absorption is an advantage, it is generally desirable that the presence of the oxide should reduce the rate of UV absorption by an amount of at least a 5%, preferably at least 10%, more preferably at least 15%, especially at least 20% and most preferably at least 40%.

It has now been found, according to the present invention, that the way in which the oxide is doped has a material effect on the efficacy of the oxide. Indeed it has now been appreciated that it is important that if the oxide is to be really effective there must be dopant on its surface which can interact with the component of the composition to be protected. For example if, in a two phase composition, the oxide is present in the aqueous phase and the component to be protected is in the organic phase there is little interaction because of the phase boundary. Thus the free radicals generated by degradation of the component cannot contact the dopant without moving from one phase to another. Although existing methods for doping in the bulk will normally also result in some dopant in or on the surface of the particle, it is possible according to the present invention to use materials which are only surface doped i.e. where there is dopant only in or on the surface of the particle. In one embodiment such materials may be used in a single phase aqueous formulation. Accordingly the present invention provides (although not dependent on the above theory) a composition suitable for veterinary, agricultural or horticultural use which comprises at least one organic veterinarally, agriculturally and/or horticulturally active compound, and titanium dioxide and/or zinc oxide which has been doped at least in or on a surface thereof with one or more other elements, typically with one i.e with only a second element. Where the particle has been bulk doped there will, in general, be dopant throughout the particle. On the other hand, where the particle has been “surface doped” (i.e. the dopant is only in or on the surface) there will be a concentration gradient such that the ratio of dopant atoms to titanium or zinc atoms at the surface or outmost “skin” of the particle is greater than the ratio in the core or centre where it may be zero.

It will be appreciated that although it will normally be the case that the bulk dopant will be the same element as the or each surface dopant (for simplicity of preparation), this need not necessarily be the case. (Of course with reduced zinc oxide there is no bulk dopant.) By this means it is possible, for example, to modify the colour of the particles. Suitable dopants for the oxide particles include manganese, which is especially preferred, e.g. Mn²⁺ but also Mn³⁺, vanadium, for example V³⁺ or V⁵⁺, chromium and iron but other metals which can be used include nickel, copper, tin, especially Sn⁴⁺, aluminium, lead, silver, zirconium, zinc, cobalt, especially Co²⁺, gallium, niobium, for example Nb⁵⁺, antimony, for example Sb³⁺, tantalum, for example Ta⁵⁺, strontium, calcium, magnesium, barium, molybdenum, for example Mo³⁺, Mo⁵⁺ or Mo⁶⁺ as well as silicon. These metals can be incorporated singly or in combinations of two or three or more. It will be appreciated that for effective bulk doping the size of the ion must be such as can readily be inserted into the crystal lattice of the particle. For this purpose Mn³⁺, vanadium, chromium and iron are generally the most effective; the ionic size of Mn²⁺ is much larger than that of Ti⁴⁺ and so there is little probability of ionic diffusion of Mn²⁺ into the TiO₂ crystal lattice. On the other hand there is no such size limitation for the elements used in surface doping; preferred surface dopants include manganese, eg. as Mn²⁺, cerium, selenium, chromium, vanadium and iron.

The optimum total amount of the second component on, and, if present in, the particle may be determined by routine experimentation but it is preferably low enough so that the particles are minimally coloured. Amounts as low as 0.1 mole % or less, for example 0.05 mole %, or as high as 1 mole % or above, for example 5 mole % or 10 mole %, can generally be used. Typical concentrations are from 0.5 to 2 mole % by weight. The mole ratio of dopant to host metal on the surface is typically from 2-25:98-75, usually 5.20:95-80 and especially 8-15:92-85. The amount of dopant at the surface can be determined by, for example, X-ray Photoelectron Spectroscopy (XPS).

The surface-doped particles can be obtained by any one of the standard processes for preparing such doped oxides and salts. These include techniques such as those described below. It will be appreciated that the dopant need not necessarily be present as an oxide but as a salt such as a chloride or a salt of an oxygen-containing anion such as perchlorate or nitrate. However bulk doping techniques will generally result in some surface doping as well and these techniques can be used in the present invention. Such techniques include a baking technique by combining particles of a host lattice (TiO₂/ZnO) with a second component in the form of a salt such as a chloride or an oxygen-containing anion such as a perchlorate or a nitrate, in solution or suspension, typically in solution in water, and then baking it, typically at a temperature of at least 300° C. Other routes which may be used to prepare the doped materials include a precipitation process of the type described in J. Mat. Sci. (1997) 36, 6001-6008 where solutions of the dopant salt and of an alkoxide of the host metal (Ti/Zn) are mixed, and the mixed solution is then heated to convert the alkoxide to the oxide. Heating is continued until a precipitate of the doped material is obtained. Further details of preparation can be found in WO 00/60994 and WO 01/40114.

It will be appreciated that such baking techniques and the like will result in dopant in the surface forming part of the crystal lattice while in other techniques the dopant will merely be adsorbed, or remain as a separate layer, on the particle surface. It is thought likely that if the dopant is to quench internally generated free radicals then it needs to be in the crystal lattice.

The rutile form of titania is known to be less photoactive than the anatase form and is therefore preferred. Zinc oxide can be in the form of reduced zinc oxide particles (i.e. particles which possess an excess of zinc ions relative to the oxygen ions).

Doped TiO₂ or doped ZnO may be obtained by flame pyrolysis or by plasma routes where mixed metal containing precursors at the appropriate dopant level are exposed to a flame or plasma to obtain the desired product.

Further discussion details of such particles can be found in WO 99/60994.

The oxide particles used in the present invention may have an inorganic or organic coating. For example, the particles may be coated with oxides of elements such as aluminium, zirconium or silicon, especially silica or, for example, aluminium silicate. The particles of metal oxide may also be coated with one or more organic materials such as polyols, amines, alkanolamines, polymeric organic silicon compounds, for example, RSi[{OSi(Me)₂}xOR¹]₃ where R is C₁-C₁₀ alkyl, R¹ is methyl or ethyl and x is an integer of from 4 to 12, hydrophilic polymers such as polyacrylamide, polyacrylic acid, carboxymethyl cellulose and xanthan gum or surfactants such as, for example, TOPO. If desired the surface doping can be carried out by a coating technique either separately or in combination with the inorganic or organic coating agent. Thus for example the undoped oxide can be coated with, say, manganese oxide along with an organic or inorganic coating agent such as silica. It is generally unnecessary to coat the oxide particles to render them hydrophilic so that for the aqueous phase the particles can be uncoated. However if the particles are to be in the organic or oily phase their surface needs to be rendered hydrophobic or oil-dispersible. This can be achieved by the application directly of, for example, a suitable hydrophobic polymer or indirectly by the application of a coating, for example of an oxide such as silica (which imparts a hydrophilic property) to which a hydrophobic molecule such as a metal soap or long chain (e.g. C₁₂-C₂₂) carboxylic acid or a metal salt thereof such as stearic acid, a stearate, specifically aluminium stearate, aluminium laurate and zinc stearate.

It should be understood that the term “coating” is not to be construed as being limited to a complete covering. Indeed it is generally beneficial for the coating not to be complete since the coating can act as a barrier to the interaction of the free radicals with the dopant on or in the surface of the particle. Thus it is preferred that the coating should be discontinuous where maximum scavenging effect is desired. However it will be appreciated that dopant on the surface can still act to quench free radicals generated within the particle in which case the coating can be continuous. Since coatings of silanes and silicones which can be polymeric or short chain or monomeric silanes are generally continuous these are generally less preferred. Thus coating with an inorganic oxide is generally preferred since these generally do not result in a complete coating on the surface of the particles.

Typical coating procedures include the deposition of silica by mixing alkali such as ammonium hydroxide with an orthosilicate, such as tetraethylorthosilicate, in the presence of the particle. Alternatively the particle can first be coated with a silane such as (3-mercaptopropyl)trimethoxy silane (MPS) and then silicate e.g. sodium silicate is added. The silane attaches to the particle surface and acts as a substrate for the silicate which then polymerises to form silica. Similar techniques can be used for other inorganic oxides.

The average primary particle size of the particles is generally from about 1 to 200 nm, for example about 1 to 150 nm, preferably from about 1 to 100 nm, more preferably from about 1 to 50 nm and most preferably from about 20 to 50 nm. Since the scavenging effect is believed to be essentially catalytic it is desirable that the particles are as small as possible to maximise their surface area and hence the area of doped material on the surface. This small size has the advantage that less dopant is needed which has the consequential advantage that any colouring effect caused by the dopant is reduced.

Where particles are substantially spherical then particle size will be taken to represent the diameter. However, the invention also encompasses particles which are non-spherical and in such cases the particle size refers to the largest dimension.

The compositions of the present invention can be single phase, either aqueous or oily or multiphase. Typical two-phase compositions comprise oil-in-water or water-in-oil formulations. For single phase compositions the oxide particles must of course be dispersible in that phase. Thus the particles are desirably hydrophilic if the composition is aqueous or hydrophobic if the composition is oil-based. However it may be possible to disperse untreated TiO₂ in the oily phase by appropriate mixing techniques. For two or multi-phase compositions the particles must be present in the phase containing the ingredient (or one of those ingredients) to be protected. It can, though, be desirable for the particles to be present in both aqueous and oily phases even if no ingredients which are to be protected are present in one of those phases. Desirably, the weight ratio of the water-dispersible particles to the oil-dispersible particles is from 1:4 to 4:1, preferably from 1:2 to 2:1 and ideally about equal weight proportions.

The present invention is applicable to any composition intended for agricultural or horticultural use which contains an organic active ingredient as well as to veterinary compositions containing an organic active ingredient, generally for topical application. Generally the active ingredient will be a biocide but it can be, for example, a plant growth promoter or regulator. Thus the compositions of the present invention are typically herbicides, fungicides, insecticides, bactericides, acaricides, molluscicides, miticides or rodenticides, which can be broad spectrum or selective. The present invention is particularly useful for fast knockdown insecticides which are badly affected by UV light. Veterinary compositions can take the form of, for example, antiseptic or wound healing preparations.

The compositions of the present invention can also be formulated for household use as with, for example, insecticides and rodenticides. Accordingly, the present invention also provides a composition suitable for household use which comprises at least one organic biocide and titanium dioxide and/or zinc oxide which has been doped with a second element and/or reduced zinc oxide.

The compositions of the present invention can contain any of the organic active ingredients currently employed for such compositions.

Suitable herbicides which can be used in the present invention include triazines, amides, in particular haloacetanilides, carbamates, toluidines (dinitroanilines), ureas, plant growth hormones, in particular phenoxy acids and diphenyl ethers. Thus herbicides which may be used include phenoxy alkanoic acids, bipyridiniums, benzonitriles with phthalic compounds, dinitroanilines, acid amides, carbamates, thiocarbamates, heterocyclic nitrogen compounds including triazines, pyridines, pyridazinones, sulfonylureas, imidazoles and substituted ureas as well as halogenated aliphatic carboxylic acids, some inorganic and organic materials and derivatives of biologically important amino acids. Specific herbicides which can be used in the present invention include 2,4-dichlorophenoxyacetic acid (2,4-D) and 2,4,5-trichlorophenoxyacetic acid (2,4,5-T). Suitable triazines include 2-chloro-, 2-methylthio-, 2-methoxy-4,6-bis-(alkylamino)-s-triazines as well as some 2-azido-substituted triazines. Typical herbicidal ureas include monuron (3-p-chlorophenyl)-1,1-dimethylurea) as well as diuron, neburon, fenuron and chloroxuron. Suitable carbamates include N-phenylcarbamate and isopropyl carbanilate (propham) and substituted derivatives thereof including isopropyl m-chlorocarbanilate (chlorpropham) as well as barban, swep, dichlormate and terbutol. Suitable thiocarbamates include EPTC, metham, vernolate, CDEC, pebulate, diallate, triallate, butylate, molinate, cycloate, thiobencarb and ethiolate. Suitable amide herbicides include solan, dicryl, propanil, dipehamid, propachlor, alachlor, CDAA, naptalam, butachlor, prynachlor and napropamide. Suitable chlorinated aliphatic acids include triochloroacetic acid (TCA), dalapon and 2,2,3-trichloropropionic acid. Suitable chlorinated benzoic acids include chloramben, DCPA, dicamba, dichlobenil and 2,3,6-TBA. Phenolic herbicides which can be used include bromoxynil, ioxynil, DNOC and dinoseb. Suitable dinitroanilines which can be used include benefin, trifluralin, nitralin, oryzalin, isopropalin, dinitramine, fluchloralin, profluralin and butralin. Suitable bypyridinium herbicides include diquat and paraquat salts and derivatives thereof.

Suitable insecticides which can be used in the present invention include nicotinoids, rotenoids, derivatives of the seeds of sabadilla and the plant ryania speciosa and pyrethroids as well as organochlorine insecticides, organophosphorus insecticides, carbamate insecticides and various insect growth regulators.

Suitable nicotinoids include nicotine sulfate and imidocloprid. The pyrethroids constitute a large group of insecticides most of which are now synthetic including resmethrin, phenothrin, cyphenothrin, empenthrin, prallethrin, permethrin, cypermethrin, alpha cypermethrin, tetramethrin and delta tetramethrin, including their isomers, especially optical isomers along with derivatives of these. Suitable organochlorine insecticides include DDT (dichlorodiphenyltrichloroethane) along with methoxychlor and perthane, as well as lindane, toxaphene, chlordane, heptachlor, aldrin, dieldrin and endrin. Suitable organophosphorus insecticides include phosphoric acid and phosphorothioic acid anhydrides, aliphatic phosphorothioate esters along with phenyl phosphorothioate esters, phenyl phosphorodithioate esters, phosphonothioate esters of phenols, vinyl phosphates, phosphorothioate esters of heterocyclic enols and of s-methyl heterocycles. Of these specific mention can be made of parathion, methyl parathion, dicapthon, chlorthion, fenitrothion, fenthion and fensulfothion along with fenchlorphos, cyanophos, propafos and temephos. Suitable carbamate insecticides which can be used include carbaryl, carbofuran, propoxur, dioxacarb, bendiocarb, mexacarbate, isoprocarb and ethiofencarb. Suitable acaricides include chlorfenethol, chlorobenzilate, dicofol, tetradifon, sulphenone, ovex, propargite, cyhexatin and dienochlor.

Some of the insecticides given above are suitable for killing rodents but other rodenticides which can be used include acute rodenticides and chronic poisons include anticoagulants; these can be stomach poisons, contact poisons or fumigants. Such anticoagulants include dicoumarol, warfarin, coumatetraly, coumachlor, difenacoum, brodifacoum, bromadiolone, pindone, diphacinone and chlorophacinone.

Insecticides which can be used in the compositions of the present invention can also be in the form of microbial agents since insects are attacked by many pathogens. These include bacterial agents, in particular bacillus microorganisms, especially bacillus thuringiensis (b.t.) strains such as b.t. aizawa, israelensis, kurstaki and tenebrionis, fungal agents, protozoa and viruses.

Suitable fungicides which can be used in the compositions of the present invention include elements such as sulphur, copper, mercury and tin along with thiocarbamate and thiurame derivatives, phthalimides and trichloromethylthiocarboximides, aromatic hydrocarbons and dicarboximides. Specific examples include ferbam, ziram, thiram, zineb, maneb and mancozeb as well as dimethylthiocarbamates and ethylene bis-dithiocarbamates. Other useful fungicides include captan, folpet, captafol and dichlofluanid. Suitable aromatic hydrocarbons include quintozene, dinocap, chloroneb, dichloran, dichlone and chlorothalonil along with oxazolidinediones such as vinclozolin, chlozolinate, hydantoin such as iprodione and succinimide such as procymidone. Other fungicides which can be used include guanidine salts such as dodine, quinones such as dithianon, quinoxalines such as chinomethionat, pyridazines such as diclomezine, thiadiazoles such as etridiazole, pyrroles such as fenpiclonil, quinolines such as ethoxyquin and triazines such as anilazine. Other fungicides which can be used include mitochondrial respiration inhibitors which are generally carboxanilides including carbox, oxycarboxin, flutolanil, fenfuram, mepronil, methfuroxam and metsulfovax. Further fungicides which can be used include microtubuline polymerization inhibitors including thiabendazole, fuberidazole, carbendazim, benomyl and thiophanate methyl. Other suitable fungicides include inhibitors of sterol biosynthesis including C-14 demethylation inhibitors such as triazoles which have a 1,2,4-triazole group attached through the 1-nitrogen to a large lipophilic group, in particular triadimefon, propiconazole, tebuconazole, cyproconazole and tetraconazole along with flusilazole which incorporates a silicon atom, myclobutanil, flutriafol and imibenconazole. Other fungicides which can be used include RNA biosynthesis inhibitors, phospholipid biosynthesis inhibitors, melanin biosynthesis inhibitors, fungal protein biosynthesis inhibitors and cell wall biosynthesis inhibitors.

The compositions of the present invention can be in liquid or solid form. Liquid compositions can be aqueous or non aqueous while solid forms include powders or dusts, granules and tablets. For rodenticides, in particular, the compositions can take the form of a bait, especially a foodstuff, for example grain, which has been treated with the rodenticide and the special oxide.

The concentration of the active ingredient in the composition can vary within a wide range but is typically 0.5 to 95, for example 1 to 50, % by weight.

A composition according to the invention preferably contains from 0.5% to 95% by weight (w/w) of active ingredient.

The compositions for agricultural or horticultural use according to the invention generally contain a carrier to facilitate application to the locus to be treated, which may for example be a plant, seed or soil, or to facilitate storage, transport or handling. The carrier may be a solid, or a liquid, as well as material which is normally a gas but which has been compressed to form a liquid.

The compositions may be in the form of, for example, emulsion concentrates, solutions, oil in water emulsions, wettable powders, soluble powders, suspension concentrates, dusts, granules, water dispersible granules, micro-capsules and gels. Other substances, such as fillers, solvents, solid carriers, surface active compounds (surfactants), and optionally solid and/or liquid auxiliaries and/or adjuvants can be present. The composition can be formulated for dispersing by, for example, spraying, atomizing, dispersing or pouring.

Solvents which may be used include aromatic hydrocarbons, e.g. substituted naphthylenes, phthalic acid esters such as dibutyl or dioctyl phthalate, aliphatic hydrocarbons, e.g. cyclohexane or paraffins, alcohols and glycols as well as their ethers and esters, e.g. ethanol, ethyleneglycol mono- and dimethyl ether, ketones such as cyclohexanone, strongly polar solvents such as N-methyl-2-pyrrolidone or γ-butyrolactone, higher alkyl pyrrolidones, e.g. n-octylpyrrolidone or cyclohexylpyrrolidone, epoxidized plant oil esters, e.g. methylated coconut or soybean oil ester and water. Mixtures can also be used.

Solid carriers, which may be used for dusts, wettable powders, water dispersible or other granules, and granules or other particles that include mineral fillers, such as silicas, calcite, talc, kaolin, montmorillonite or attapulgite. The physical properties may be improved by addition of highly dispersed silica gel or polymers. Carriers for granules may be porous material, e.g. pumice, kaolin, sepiolite, bentonite; non-sorptive carriers may be calcite or sand.

The compositions can be formulated as concentrates which can subsequently be diluted by the user before application. The presence of small amounts of a carrier which is a surfactant facilitates this process of dilution. Thus, preferably the compositions according to the invention preferably contain a surfactant. For example, the composition may contain two or more carriers, at least one of which is a surfactant. Such surfactants may be nonionic, anionic, cationic or zwitterionic.

The compositions of the invention may for example be formulated as wettable powders, water dispersible granules, dusts, granules, solutions, emulsifiable concentrates, emulsions, suspension concentrates and aerosols. Wettable powders usually contain 5 to 90% w/w of active ingredient and 3 to 10% w/w of dispersing and/or wetting agent and, where desirable, 0 to 10% w/w of stabilizer(s) and/or other additives such as penetrants or stickers. Dusts are usually formulated as a dust concentrate having a similar composition to that of a wettable powder but without a dispersant. Water dispersible granules are usually prepared to have a size from 0.15 mm to 2.0 mm and contain 0.5 to 90% w/w active ingredient and 0 to 20% w/w of additives such as stabilizers, surfactants, slow release modifiers and binding agents. Emulsifiable concentrates usually contain, in addition to a solvent or a mixture of solvents, 1 to 80% w/v active ingredient, 2 to 20% w/v emulsifiers and 0 to 20% w/v of other additives such as stabilizers, penetrants and corrosion inhibitors. Suspension concentrates usually contain 5 to 75% w/v active ingredient, 0.5 to 15% w/v of dispersing agents, 0.1 to 10% w/v of suspending agents such as protective colloids and thixotropic agents, 0 to 10% w/v of other additives such as defoamers, corrosion inhibitors, stabilizers, penetrants and stickers, and water or an organic liquid in which the active ingredient is substantially insoluble; certain organic solids or inorganic salts may be present dissolved in the formulation to assist in preventing sedimentation and crystallization or as antifreeze agents for water.

The Example given above in connection with the first embodiment also illustrates this embodiment.

1. A particle of TiO₂ or ZnO which has been doped with one or more other elements such that the concentration of dopant in a surface of the particle is greater than that at a core of the particle.

2. A particle according to claim 1 which is coated with a discontinuous layer of hydrophilic or hydrophobic material.

3. A particle according to claim 2 which is coated with hydrophobic polymer.

4. A particle according to claim 2 which is coated first with an oxide of aluminium, zirconium or silicon and then with a long chain carboxylic acid salt.

5. A process for preparing a particle as claimed in claim 1 which comprises placing a particle of TiO₂ or ZnO in contact with a solution or suspension of a salt of the dopant for a time insufficient for the concentration of dopant salt in the core of the particle to reach that at its surface and then baking the resulting particle.

6. A process according to claim 5 wherein the particle is baked at a temperature of at least 500° C.

7. A particle according to claim 1 that is prepared by placing a particle of TiO₂ or ZnO in contact with a solution or suspension of a salt of the dopant for a time insufficient for the concentration of dopant salt in the core of the particle to reach that at its surface and then baking the resulting particle.

8. A UV sunscreen composition suitable for cosmetic or topical pharmaceutical use which comprises: (a) one ore more organic components which are photosensitive and/or which are susceptible to degradation by another ingredient of the composition and/or by undoped TiO₂ and/or by undoped ZnO; and (b) TiO₂ and/or ZnO which has been surface doped with one or more other elements.

9. A composition according to claim 8 which is an aqueous formulation and the TiO₂ and/or ZnO is only surface doped.

10. A composition according to claim 8 which is an oily formulation.

11. A composition according to claim 8 which is an oil-in-water or water-in-oil formulation.

12. A composition according to claim 11 wherein the TiO₂ and/or ZnO is present in both phases.

13. A composition according to claim 8 wherein the TiO₂ and/or ZnO is coated with a discontinuous layer of hydrophilic or hydrophobic material.

14. A composition according to claim 13 wherein the TiO₂ and/or ZnO is coated with a hydrophobic polymer.

15. A composition according to claim 13 wherein the TiO₂ and/or ZnO is coated first with an oxide of aluminium, zirconium or silicon and then with a long chain carboxylic acid salt.

16. A composition according to claim 8 wherein one or more of the said organic components is a UV sunscreen agent.

17. A composition according to claim 16 wherein the organic sunscreen agent absorbs UV light in the UVA region.

18. A composition according to claim 16 wherein the organic sunscreen agent is a paraaminobenzoic acid, ester or derivative thereof, a methoxy cinnamate ester, a benzophenone, a dibenzoylmethane, an alkyl-p-p-phenyl acrylate, a triazine, a camphor derivative, an organic pigment, a silicone based sunscreen agent or 2-phenylbenzimidazoyl-5 sulphonic acid or phenyldibenzimidazoyl sulphonic acid.

19. A composition according to claim 8 which contains one or more of a fatty substance, organic solvent, silicone, thickener, demulsant, UVB sunscreen agent, antifoaming agent, moisturising agent, perfume preservative, surface activation filler, sequestrant, anionic, cationic, nonionic or amphoteric polymer, propellant, alkalising or acidifying agent, colourant or metal oxide pigment.

20. A composition according to claim 8 which is a sunscreen.

21. A method for reducing the concentration of one or more organic UV sunscreen agents or other ingredient which is photosensitive and/or is degraded by another ingredient in a UV sunscreen composition comprising incorporating into the composition a doped TiO₂/ZnO as defined in claim 1 to reduce the concentration of one or more organic UV sunscreen agents or other ingredient which is photosensitive and/or is degraded by another ingredient in the UV sunscreen composition.

22. A process for increasing the effectiveness of an organic UV sunscreen composition which comprises one or more components which are photosensitive and/or are susceptible to degradation by another ingredient of the composition and/or by undoped TiO₂ and/or by undoped ZnO, which process comprises incorporating into the composition a doped TiO₂/ZnO as defined in claim 1.

23. A process for reducing the production of a toxic compound in a UV sunscreen composition which process comprises incorporating therein doped TiO₂ and/or ZnO as defined in claim 1.

24. A composition which comprises an amount of one or more organic or inorganic components which are photosensitive and/or which are degraded by another ingredient of the composition and an amount of TiO₂ and/or ZnO which has been doped at least on or in a surface thereof with one or more other elements.

25. A composition according to claim 24 which has a rate of deterioration of a UV light-sensitive physical factor at least 5% less than that of a composition having the same formulation except that it does not contain the said TiO₂ and/or ZnO which has been doped with a second element.

26. A composition according to claim 25 wherein the physical factor is tensile strength.

27. A composition according to claim 25 wherein the physical factor is colour.

28. A composition according to claim 8 which contains TiO₂ and/or ZnO which has not been doped, optionally as TiO₂ and/or ZnO particles which have not been doped.

29. A composition according to claim 28, wherein the said TiO₂ and/or ZnO is present as pigment.

30. A composition according to claim 8 in the form of a coating on and/or an additive in a polymeric material, which material is thermoplastic, or thermosetting or photosensitive.

31. A composition according to claim 8 which is in the form of a three dimensional article, or is in the form of a film, or is in the form of a photographic film, or is in the form of a coating composition, or is in the form of a paint or varnish.

32. A self-supporting polymeric composition intended to protect a composition adjacent thereto from the adverse effects of light which comprises TiO₂ and/or ZnO which has been doped at least in or on a surface thereof with one or more other elements or reduced ZnO.

33. A composition according to claim 32 wherein the TiO₂ and/or ZnO is present in a surface layer.

34. A composition according to claim 33 wherein a non-surface layer thereof is not wood.

35. A composition according to claim 33 wherein a non-surface layer thereof is synthetic.

36. A varnish composition which comprises TiO₂ and/or ZnO which has been doped at least in or on a surface thereof with one or more other elements or reduced ZnO.

37. A composition according to claim 32 which has a rate of deterioration of a UV light-sensitive physical factor at least 5% less than that of a composition having the same formulation except that it does not contain the said TiO₂ and/or ZnO which has been doped with a second element.

38. A method for reducing the concentration of one or more light stabilisers in a polymeric composition comprising incorporating into the composition a surface doped TiO₂/ZnO as defined in claim 1 to reduce the concentration of one or more light stabilisers in the polymeric composition.

39. A method for reducing the rate of deterioration of a light-sensitive physical factor in a polymeric composition comprising incorporating into the composition a surface doped TiO₂/ZnO as defined in claim 1 to reduce the rate of deterioration of a light-sensitive physical factor in the polymeric composition.

40. A process for improving stability of a physical factor of a polymeric composition, which comprises one or more components which are photosensitive and/or are degraded by another ingredient of the composition which process comprises incorporating into the composition a surface doped TiO₂/ZnO as defined in claim 1.

41. A composition suitable for veterinary, agricultural or horticultural use which comprises at least one organic veterinarally, agriculturally and/or horticulturally active compound, and titanium dioxide and/or zinc oxide which has been doped at least in or on a surface thereof with one or more other elements.

42. A composition according to claim 41 wherein the active compound is a herbicide, fungicide, insecticide, acaricide, miticide or rodenticide.

43. A composition suitable for household use which comprises at least one organic biocide, and titanium dioxide and/or zinc oxide which has been doped at least in or on a surface thereof with one or more other elements.

44. A particle according to claim 1 wherein the dopant is manganese, selenium, cerium, chromium, vanadium or iron.

45. A particle according to claim 1 wherein the dopant is Mn²⁺ or other manganese, or is V⁴⁺.

46. A particle according to claim 1 wherein the dopant is present in an amount from 0.05% to 10 mole %.

47. A particle according to claim 46 wherein the dopant is present in an amount from 0.5 to 2 mole % by weight.

48. A particle according to claim 1 in which the doped oxide is doped titanium dioxide.

49. A particle according to claim 1 wherein the titanium dioxide is in rutile form.

50. A composition according to claim 8 which contains reduced zinc oxide.

51. A composition according to claim 8 which comprises 0.5 to 20 mole % by weight of the doped titanium dioxide and/or zinc oxide.

52. A particle according to claim 1 wherein the doped or reduced oxide has a particle size from 1 to 200 nm, preferably 1 to 100 nm; or from 100 to 500 nm.

53. A composition according to claim 42 wherein the active compound is an insecticide.

54. A composition according to claim 8 which contains one or more of a filler, organic solvent or surfactant.

55. A composition according to claim 8 which is in the form of an aqueous or non-aqueous liquid, a powder, granules or tablet.

56. A method for reducing the concentration of one or more veterinarally, agriculturally and/or horticulturally active compounds in a composition suitable for veterinary, agricultural, horticultural or household use comprising incorporating into the composition a surface doped TiO₂/ZnO as defined in claim 1 to reduce the concentration of one or more veterinarally, agriculturally and/or horticulturally active compounds in the composition suitable for veterinary, agricultural, horticultural or household use.

57. A method for increasing the shelf life of one or more veterinarally, agriculturally and/or horticulturally active compounds in a composition suitable for veterinary, agricultural, horticultural or household use comprising incorporating into the composition a surface doped TiO₂/ZnO as defined in claim 1 to increase the shelf life of one or more veterinarally, agriculturally and/or horticulturally active compounds in the composition suitable for veterinary, agricultural, horticultural or household use.

58. A process for increasing the effectiveness of a composition suitable for veterinary, agricultural, horticultural or household use which process comprises one or more organic veterinarally, agriculturally or horticulturally or household active compounds, which comprises incorporating into the composition a surface doped TiO₂/ZnO as defined in claim 1.

59. A process for treating an agricultural or horticultural species at a locus which process comprises treating the locus with a composition as claimed in claim 41.

60. A particle according to claim 1 in which the mole ratio of dopant to host metal at the surface is 2-25 to 98-75.

61. A particle according to claim 60, in which the mole ratio of dopant to host metal at the surface is 8-75 to 92-25.

62. A particle according to claim 1 in which the concentration of dopant in a surface of the particle is greater than in the bulk of the particle.

63. A particle according to claim 1 in which there is no dopant at the core of the particle and/or in the bulk of the particle.

64. A particle according to claim 1 in which a dopant is present in the bulk of the particles, and wherein the bulk dopant is different from the or each surface dopant.

65. (canceled)
